(19) United States
(12) Patent Application Publication
(10) Pub. No.: US 2013/0210652 A1
(43) Pub. Date: Aug. 15, 2013

(54) NANOTECHNOLOGY ENABLED POINT OF CARE TESTING DEVICE

(71) Applicants: Krishnan Chakravarthy, Williamsville, NY (US); Siddhartha Kamisetti, Cleveland, OH (US); Himanshu Subhash Amin, Solon, OH (US)

(72) Inventors: Krishnan Chakravarthy, Williamsville, NY (US); Siddhartha Kamisetti, Cleveland, OH (US); Himanshu Subhash Amin, Solon, OH (US)

(73) Assignee: NANOAXIS, LLC, Clarence, NY (US)

(21) Appl. No.: 13/665,986

(22) Filed: Nov. 1, 2012

Related U.S. Application Data
(60) Provisional application No. 61/554,145, filed on Nov. 1, 2011.

(51) Int. Cl. G01N 33/58; C12Q 1/26

(52) U.S. Cl. CPC G01N 33/588; C12Q 1/26
USPC 506/9; 506/39; 506/11; 506/12

(57) ABSTRACT

The present invention relates to an apparatus to detect biological parameters in the analysis of a sample, specimen, or assay. The system includes a device for determining one or more values for one or more measurable characteristics of a sample. In an aspect, the device can be configured to operate on any of a mobile device, tablet computer, laptop computer, desktop computer, digital pen, medical testing tool or electronic device. In another aspect, the device can comprise a synchronization component that synchronizes the device to a network system.

### CROSS REFERENCED TO RELATED APPLICATION

This application claims the benefit of U.S. Provisional Patent Application Ser. No. 61/554,145 filed Nov. 1, 2011 and titled “Point of Care Testing Device”, which is incorporated by reference herein in its entirety.

### FIELD

The present invention relates to a device to detect and measure biological targets in a biological sample. The device can determine one or more values for one or more measurable characteristics of a sample.

### BACKGROUND

The Comprehensive Metabolic Panel (CMP) is a broad screening tool for various medical problems (e.g. diabetes, liver disease, kidney disease, etc.) or verification tool for a subject's health. Current methods of performing CMP screening involves a series of blood tests, laboratory tests, screenings, protein monitoring, electrolyte detection, and other tests to individually check liver function, glucose levels, calcium concentrations, and other such subject profiling. CMP tests and other tests to measure the presence of a biological substance (e.g. urea, glucose, sodium, electrolytes, etc.) are time consuming, burdensome, costly, labor intensive, inefficient and lacking specificity, and often dependent on patient compliance, and are often conducted in a laboratory at a location separate from the location where the subject resides. Additionally, the accuracy of current tests are questionable. For instance, measuring glucose levels with a glucometer can result in a ±20% error range, which is a real concern for many health care practitioners. Such errors are due in part to the change in hemocrit values resulting from the pathological condition of a patient. These patient tests play an important role in the detection and diagnosis of health ailments, but there is a need to overcome the issues with current diagnostic tests.

### SUMMARY

The following presents a simplified summary of the disclosure in order to provide a basic understanding of some aspects of the disclosure. This summary is not an extensive overview of the disclosure. It is intended to neither identify key or critical elements of the disclosure nor delineate any scope particular embodiments of the disclosure, or any scope of the claims. Its sole purpose is to present some concepts of the disclosure in a simplified form as a prelude to the more detailed description that is presented later

In accordance with one or more embodiments and corresponding disclosure, various non-limiting aspects are described in connection with a nanotechnology-enabled point of care device. In accordance with a non-limiting embodiment, in an aspect, a device is provided in connection with a cartridge component that stores nanomaterial labels paired to biological materials in N wells, whereby each well stores one or more nanomaterial labels paired to one or more biological materials; wherein N is an integer; a dispenser component that dispenses a biological sample into each respective well; an excitation component that emits energy from an excitation source to excite the nanomaterial label in the absence of the biological sample and excites the nanomaterial label in the presence of the biological sample; a detection component that detects a relative change in energy emitted from the nanomaterial label paired to the biological material in the absence of the biological sample as compared to the energy emitted from the nonmaterial label paired to a biological material in the presence of a biological target present in the biological sample; an analysis component that analyzes data related to the change in energy emitted from the nanomaterial label paired to the biological material as compared to a standard curve for the energy emitted from the nanomaterial label paired to a the biological material in the presence of various concentration levels of the biological target; a generation component that generates a medical diagnostic report based in part on the analyzed data; and a display component that displays the medical diagnostic report to a device user.

The disclosure further discloses a method, comprising using a processor to execute computer executable instructions stored in a memory to perform the following acts: storing nanomaterial labels paired to biological materials in N wells, whereby each well stores one or more nanomaterial labels paired to one or more biological materials; wherein N is an integer; dispensing a biological sample into each respective well; emitting energy from an excitation source to excite the nanomaterial label in the absence of the biological sample and excites the nanomaterial label in the presence of the biological sample; detecting a relative change in energy emitted from the nanomaterial label paired to the biological material in the absence of the biological sample as compared to the energy emitted from the nanomaterial label paired to a biological material in the presence of a biological target present in the biological sample; analyzing data related to the change in energy emitted from the nanomaterial label paired to the biological material as compared to a standard curve for the energy emitted from the nanomaterial label paired to a the biological material in the presence of various concentration levels of the biological target; generating a medical diagnostic report based in part on the analyzed data; displaying the medical diagnostic report to a device user.

The following description and the annexed drawings set forth certain illustrative aspects of the disclosure. These aspects are indicative, however, of but a few of the various ways in which the principles of the disclosure may be employed. Other advantages and novel features of the disclosure will become apparent from the following detailed description of the disclosure when considered in conjunction with the drawings.

### BRIEF DESCRIPTION OF THE FIGURES

FIG. 1 illustrates an example non-limiting nanotechnology enabled point of care device.

FIG. 2 illustrates an example non-limiting nanotechnology enabled point of care device.

FIG. 3 illustrates an example non-limiting nanotechnology enabled point of care device.

FIG. 4 illustrates an example non-limiting cartridge component.

FIG. 5 illustrates an example non-limiting individual well of a cartridge component.

FIG. 6 illustrates an example non-limiting bonding of a nanomaterial label to a biological material to permit multiplexing applications.

FIG. 7 illustrates an example non-limiting mechanism for determining glucose concentration within a well.

FIG. 8 illustrates an example non-limiting mechanism for determining potassium concentration in a sample.

FIG. 9 illustrates an example non-limiting mechanism for determining bicarbonate concentration.

FIG. 10 illustrates an example non-limiting mechanism for determining sodium concentrations in a sample.

FIG. 11 illustrates an example non-limiting mechanism for determining urea concentration in a sample.

FIG. 12 illustrates an example non-limiting mechanism for detecting PH in a sample.

FIG. 13 illustrates an example non-limiting mechanism for determining the presence of a sexually transmitted disease in a sample.

FIG. 14 illustrates an example non-limiting embodiment of a nanotechnology enabled point of care device.

FIG. 15 illustrates an example non-limiting embodiment of fluorescence spectra of quantum dot's (QD's) with various ligands. The fluorescence spectra of water-soluble CdSe/ZnS quantum dots coated with chemically reduced Bovine Serum Albumin (BSA) (curve 2), standard BSA (curve 3), and succinylated BSA (curve 4). Without BSA treatment, the fluorescence spectra of water-soluble dots (curve 5) showed a 10 nm redshift in wavelength and were only ⅕ as bright as that of the original dots dissolved in chloroform (curve 1). With reduced BSA layer, both the fluorescence intensity and the spectral wavelength were restored to the original values.

FIG. 16 illustrates an example non-limiting embodiment of the mechanism of glucose sensing using bienzyme hybrid system.

FIG. 17 (a) illustrates an example non-limiting embodiment of the calibration of CdTe-GOD system by Cao et. Al, 2008. The relative fluorescence intensity changes after addition of different concentrations of glucose to the sensing solution.

FIG. 17 (b) illustrates the calibration of CdTe-God system by Cao et. Al., 2008. Lineweaver-Burke plots of the GOx enzyme-glucose reaction.

FIG. 18 illustrates an example non-limiting embodiment of a comparison of sensitivity with different capping agents on the nanocrystals. PL spectra represented comparison of the six sequential scans of GSH-capped QD's and MSA-capped QD's with the addition of 1 mM h2o2. Scan rate, 500 nm/min; scan delays, 3 min.

FIG. 19(A) illustrates an example non-limiting embodiment of a 5(A) illustrates the Glucose biosensor based on nanocomposite film. The scheme of sensing assembly: (a) shows the top 3 bilayers of PAH/GOD. (b) shows the 3 bilayers of PAH/PSS, and (c) shows 12 bilayers of PAH/CdTe nanocrystals.

FIG. 19(B) illustrates an example non-limiting embodiment of the glucose biosensor based on nanocomposite film. The time-dependent fluorescent changes recorded at 630 nm upon the interaction of (PAH/CdTe)12(PAH/PSS)3(PAH/GOD)3 multilayer with variable concentrations of glucose: (a) 2, (b) 4, (c) 6, (d) 8, (e) 12, (f) 16, (g) 20, and (h) 40 mM.

FIG. 19(C) illustrates an example non-limiting embodiment of illustrates the absolute quenching rate of the photoluminescence intensity taken within 5 minutes as a function of glucose concentration. F0 and Fm represent the photoluminescence intensity of (PAH)/CdTe)12(PAR/PSS)3(PAX/GOD)3 multilayer at emission maximum in the absence (F0) and presence (Fm) of glucose.

FIG. 20 illustrates an example non-limiting embodiment of glucose sensing by nanocrystals immobilized in smart microgels. Reversible fluorescence quenching and antiquencing of CdS nanocrystals embedded in the interior of p (NIPAM-AAm-PBA) microgels in response to the change in glucose concentration. (a) Characteristic PL response of the p(MIPAM-AAm-PBA)-CdS hybrid microgels at 638 nm in the presence of D-glucose at pH=8.8.

FIG. 21 illustrates an example non-limiting embodiment of the mechanism scheme of LDH and glucose sensing. The schematic principle for assay of NAD (A) and the activity of the LDH (B) based on the electron transfer of nanocrystals and the biochemical reaction (left).

FIG. 22 illustrates an example non-limiting graph of urea sensing using CdSe—ZnS.

FIG. 23 illustrates an example non-limiting graph of urea calibration curves reported by Duong and Rhee (2008). The linear plot for urea sensing using the QD-entrapped sol-gel membrane (left). The urea sensing using the urease immobilized sol-gel membrane (middle). The sensing of urea using the double layer of QD-trapped and urease immobilized sol-gel membrane (right).

FIG. 24 illustrates an example non-limiting embodiment of sodium sensing using QD's. (a) is the assembly of sodium nano-optode, (b) is the absorption spectra of chromoionophore (gray lines) and the emission spectra of the QD (red line).

FIG. 25 illustrates an example non-limiting embodiment of response curves for sodium biosensing. (a) is the experimental response of ion-selective QD's to sodium concentrations. (b) is the calibration curve of ratiometric ISQD.

FIG. 26 illustrates an example non-limiting embodiment of the effect of pH on QD Fluorescence in view to study enzyme kinetics. The emission spectrum of QD's to the variation of pH. A to H are 8.0, 7.5, 7.0, 6.5, 6.0, 5.5, 5.0, 4.5, respectively. Inset shows the variation of the FL intensity of QD's at 562 nm at various pH.

FIG. 27 illustrates an example non-limiting effect of pH on QD fluorescence in view of virus detection. (A) is the variation of fluorescence with change in pH. (B) is the Peak fluorescence intensity of each curve plotted against pH.

FIG. 28 illustrates an example non-limiting embodiment of the mechanism for glucose biosensing. The H2O2 released during Glucose oxidase enzyme, acts as an electron acceptor, thus quenching the fluorescence of QD's.

FIG. 29 illustrates an example non-limiting embodiment of the conjugation of Glucose Oxidase (GOD) to QD's using EDC and NHS as the cross-linkers, giving an amide linkage between the QD and GOD.

FIG. 30 illustrates equations that explain the estimation of Enzyme Activity in Units of GOD per mg of QD's.

FIG. 31 illustrates a table to explain the properties of CdSe, CdS and ZnS semiconductors used.

FIG. 32 illustrates TEM micrographs of QD's. TEM microsgraphs for CdSe—ZnS (MAA) (A), (MPA (B), GSH (C), CdSe—CdS MAA (D) MPA (E), GSH (F) and CdS—ZnS MAA (G), MPA (H), GSH (I).

FIG. 33 illustrates HRTEM micrographs of QD's. HRTEM micrographs and ED patters for CdSe—ZnS (A and D), CdSe—CdS (B and F) and CdS—ZnS (C and F). Scale bar=5 nm for all the HRTEM micrographs and Scale bar=10000000 nm for all the ED patterns.

FIG. 34 illustrates size distribution and analysis of QD-ligand systems. Absorbance spectra for CdSe—ZnS, CdSe—CdS and CdS—ZnS for the three ligands MAA, MPA, and GSH. Comparison of mean particle size (nm) for the nine systems from TEM and Absorbance.

FIG. 35 illustrates fluorescence spectra and its response to H2O2. fluorescence spectra for CdSe—ZnS (MAA) (A), MPA (B), GSH (C), CdSe—CdS (MAA) (D), MPA (E), GSH (F), and CdS—ZnS (MAA) (G), MPA (H), GSH (I). The spectra are recorded for varying concentrations of H2O2 (as shown in side bar) at an excitation wavelength of 390 nm. The S value shown under each panel is the slope of Relative Intensity (I/IO) versus H2O2 concentration. The sensitivity Index (SI is defined here as the modulus of the slope S.

FIG. 36 illustrates a table of Enzyme activity for the nine QD-ligand systems estimated after 0, 2 and 4 weeks, with samples kept at room temperature.

FIG. 37 illustrates an evaluation of QD-ligand systems for performance as biosensors. The sealed values, (as per equation 6) for the three parameters, (SI, EA and ESC) are shown at the top of each system.

FIG. 38 illustrates relative fluorescence of the QD-ligand systems to various concentrations of glucose.

FIG. 39 illustrates modified lineweaver-burke plots of the QD-ligand systems.

FIG. 40 illustrates relative fluorescence of the QD-ligand system (uncentrifuged) to various concentrations of glucose.

FIG. 41 illustrates modified Lineweaver-Burke plotsof the QD ligand systems (uncentrifuged).

FIG. 42 illustrates Linear Fit model of glucose estimation.

FIG. 43 illustrates a table which shows the testing the QD-ligand-enzyme system with real plasma samples.

FIG. 44 illustrates an example non-limiting embodiment of a methodology for storing, dispensing, emitting energy, detecting, analyzing, generating, and displaying a diagnostic report in connection with a point of care device.

FIG. 45 illustrates an example non-limiting embodiment of a methodology for storing, dispensing, emitting energy, detecting, analyzing, generating, displaying a diagnostic report, and synching in connection with a point of care device.

FIG. 46 is a block diagram representing an exemplary non-limiting networked environment in which the various embodiments can be implemented.

FIG. 47 is a block diagram representing an exemplary non-limiting computing system or operating environment in which the various embodiments may be implemented.

### DETAILED DESCRIPTION

### Overview

The innovation is now described with reference to the drawings, wherein like reference numerals are used to refer to like elements throughout. In the following description, for purposes of explanation, numerous specific details are set forth in order to provide a thorough understanding of this innovation. It may be evident, however, that the innovation can be practiced without these specific details. In other instances, well-known structures and components are shown in block diagram form in order to facilitate describing the innovation.

By way of introduction, the subject matter disclosed in this disclosure relates to a point of care device to detect and measure biological targets in a biological sample. The point of care device offers the ability to measure a variety of medical screening tests including, but not limited to, a comprehensive metabolic panel of a subject simultaneously at a patient's bedside. The bedside analysis of this clinical information eliminates the need for time consuming and labor intensive laboratory tests while allowing a physician to have sometimes critical medical information about a patient immediately.

The device utilizes the advanced technologies of quantum dots to measure numerous biological targets and concentrations of such targets in a biological sample, such as blood simultaneously. Previously, multiple blood tests and screening tests (e.g. BMP/electrolytes, arterial blood gas, alveolar gas, calcium, magnesium, potassium, renal tests, urinalysis, protein, liver function tests, etc.) would have to be performed separately to determine the concentration of lipids, electrolytes, and other such biological substances in a subject. Quantum dots are luminescent semiconductor nanoparticles which emit light when activated by an energy source such as UV light. A great advantage of quantum dots are its use in multiplexing applications whereby each quantum emission wavelength can be detected by a reader, such as a UV spectrophotometer, as a unique spectral signature.

In an embodiment, multi-leg luminescent nanoparticles are useful for multiplexing applications in that by adjusting the leg width, leg length, number of legs, or base size; several unique multi-leg luminescent nanoparticles can be synthesized, each comprising a unique spectral signature. By utilizing multiplexing features, the device can associate a unique spectral signature with the presence of a unique biological target in order to detect several biological targets in a single given biological sample simultaneously and in a nominal amount of time (e.g. minutes rather than hours or days).

### Example Point of Care Device for Detecting and Measuring Several Biological Targets in a Biological Sample

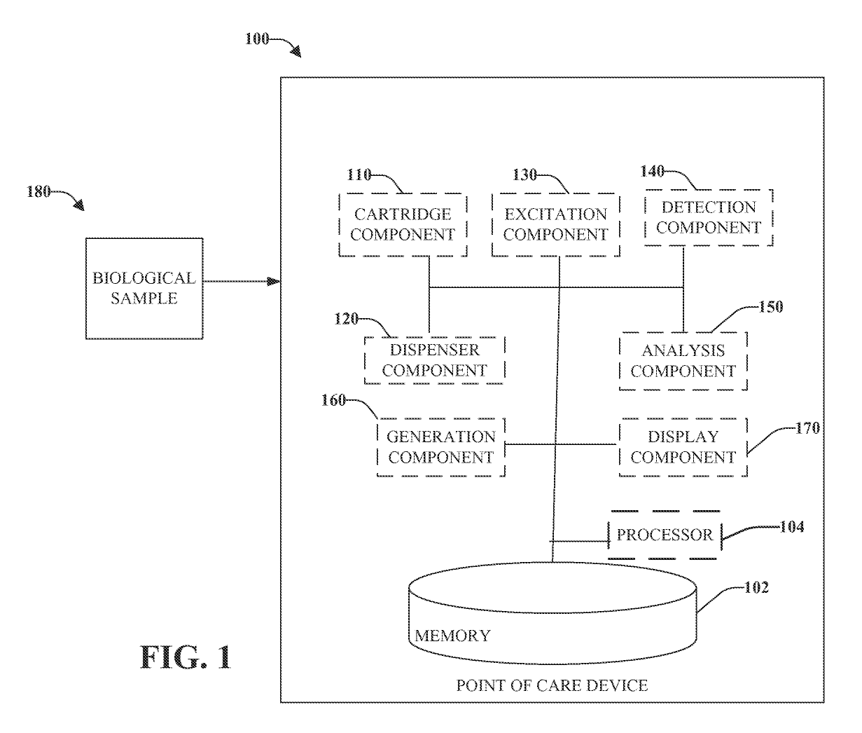

Referring now to the drawings, with reference initially to FIG. 1, point of care device 100 is shown that facilitates detection of numerous biological targets simultaneously. Aspects of the device, systems, or processes explain this disclosure can constitute machine-executable component embodied within machine(s), e.g., embodied in one or more computer readable mediums (or media) associated with one or more machines. Such component, when executed by the one or more machines, e.g. computer(s), computing device(s), virtual machine(s), etc. can cause the machine(s) to perform the operations described. Point of care device can include memory 102 for storing computer executable components and instructions. A processor 104 can facilitate operation of the computer executable components and instructions by the point of care device 100.

In an embodiment, point of care device 100 employs a cartridge component 110, a dispenser component 120, an excitation component 130, a detection component 140, an analysis component 150, a generation component 160, and a display component 170. Cartridge component 110 stores nanomaterial labels paired to biological materials in N wells, whereby each well stores one or more nanomaterial labels paired to one or more biological materials; wherein N is an integer. Dispenser component 120 dispenses a biological sample into each respective well. Excitation component 130 emits energy from an excitation source to excite the nanomaterial label in the absence of the biological sample and excites the nanomaterial label in the presence of the biological sample.

Detection component 140 detects a relative change in energy emitted from the nanomaterial label paired to the biological material in the absence of the biological sample as compared to the energy emitted from the nanomaterial label paired to a biological material in the presence of a biological target present in the biological sample. Analysis component 150 analyzes data related to the change in energy emitted from the nanomaterial label paired to the biological material as compared to a standard curve for the energy emitted from the nanomaterial label paired to a the biological material in the presence of various concentration levels of the biological target. Generation component 160 generates a medical diagnostic report based in part on the analyzed data. Display component 170 displays the medical diagnostic report to a device user.

In an aspect, cartridge component 110 stores nanomaterial labels paired to biological materials in N wells, whereby each well stores one or more nanomaterial labels paired to one or more biological materials; wherein N is an integer. A nanomaterial label is a luminescent nanoparticle ranging in size from 0.001 nm to 999.99 nm capable of pairing to a biological material and a biological target. Furthermore, a nanomaterial label comprises the ability to exhibit a change in absorption of energy (e.g. electromagnetic radiation, ultraviolet light, particle beam, and other such energy sources) and able to emit a unique spectral signature represented as a narrow emission wavelength band. Additionally, the nanomaterial label is a luminescent nanoparticle capable of excitement from an energy source (e.g. electromagnetic radiation, ultraviolet light, particle beam, etc.).

The nanomaterial label can be a quantum dot such as a spherical quantum dot, tetrapod quantum dot, quantum dot heterostructure, multi-leg luminescent nanomaterial, multi-branched luminescent nanomaterial, snowflake-shaped quantum dot, teardrop-shaped quantum dot, disk-shaped quantum dot, cube-shaped quantum dot, or star-shaped quantum dot. In an aspect, the nanomaterial label can be a multi-leg luminescent nanoparticle (“MLN”). An MLN comprises a base and one or more legs protruding from the base.

In an aspect, a base describes the component of the MLN from which one or more legs extend. In an aspect, a one or more leg describes one or more protrusions from the base of the MLN. Each MLN has ‘x’ legs extending from a base material whereby “n” in an integer. In an aspect, the addition of each new leg, allows the MLN to be detected by a reader (e.g. UV spectrophotometer, flow cytometer, etc.) as a new spectral signature that is different and identifiable from any MLN that lacks the same number of legs. For instance, and MLN whereby x=3 comprises three legs extending from the base, however an MLN whereby x=5 comprises five legs extending from the base. The MLN whereby x=3 has a unique spectral signature than the MLN whereby n=5. Furthermore, the unique spectral signature allows for each respective MLN to be identified individually by detection due to the differentiability of the spectral signature from other spectral signatures.

In an aspect, each leg comprises a leg length and a leg width, each of which can be respectively adjusted for each MLN. A leg length describes the distance from the centroid of the MLN to the tip end point of a leg whereby the leg length is a length greater than or equal to 0.001 nm and less than or equal to 999.999 nm. Each MLN leg has a leg width which is the distance from the centroid of the MLN to the side point of a leg whereby the leg width is a length greater than or equal to 0.001 nm and less than or equal to 999.999 nm. Each leg length and each leg width can be adjusted to a distance greater than or equal to 0.001 nm and less than or equal to 999.99 nm and each leg on a particular MLN can be of different leg length or leg width whereby each unique leg width and each unique leg length respectively present a unique spectral signature on a reader.

Furthermore, in an aspect, an MLN has a base length which is the distance from the centroid of the MLN to the base edge wherein the distance from the centroid to the base edge can be greater than or equal to 0.001 and less than or equal to 999.999 nm in distance and can be adjusted for each unique MLN. Additionally, in an aspect, an MLN comprises a base edge which is the point where each leg junctures with the base. Each leg can extend from a different base edge point thereby allowing for multiple base edges regions for each MLN.

In another aspect, each MLN may comprise legs of different leg lengths, legs of the same length or a mixture of legs of the same length, legs of different leg length, legs of the same leg width's, legs of different leg width's, MLN's of different base lengths, or MLN's of different base edge's. Each combination of leg length, leg width, base length or number of legs characterizing a respective MLN allows for the synthesis of numerous MLN's, each with a different spectral signature output thereby resulting in an detection of several uniquely identifiable MLN's. This simultaneous identification of several unique MLN's allows for multiplexing in the point of care device whereby multiple biological targets can be labeled by unique MLN's (e.g. an MLN with a unique spectral signature) and detected. Furthermore, several MLN features (number of legs, leg length, leg width, base length, etc.) can be adjusted to allow for hundreds (and in some cases thousands) of unique MLN to respectively identify hundreds of unique biological targets simultaneously.

The quantum dot, such as an MLN, can be water-soluble (e.g. synthesized to be hydrophilic), have one or more pairing moieties on the external surface to provide surface chemistries, and surface physiochemical properties enabling the quantum dot to pair to biological materials or biological targets. A pairing moiety can be any one or more of a thiol, F127COOH, alkyl group, propyl group, N-(3-aminopropyl)-3-mercapto-benzamide, 3-aminopropyl-trimethoxysilane, 3-mercaptopropyltrimethoxysilane, 3-maleimidopropyl-trimethoxysilane, or 3-hydrazidopropyl-trimethoxysilane, diacetylenes, acrylates, acrylamides, vinyl, and styryl. In an aspect, the pairing moiety is any one of a thiol moiety, F127COOH, alkyl group, propyl group, N-(3-aminopropyl)-3-mercapto-benzamide, 3-aminopropyl-trimethoxysilane, 3-mercaptopropyltrimethoxysilane, 3-maleimidopropyl-trimethoxysilane, or 3-hydrazidopropyl-trimethoxysilane, diacetylenes, acrylates, acrylamides, vinyl, styryl, or other such functional group. In another aspect, pairing moieties can be chemical groups, or any combination of chemical groups, including, but not limited to, amino groups, carboxyl groups, azide groups, alkyne groups, hydrazine groups, aldehyde groups, aminooxy groups, ketone groups, maleimide groups, thiol groups, or other such chemical groups. Furthermore, pairing moieties can be diacetylenes, acrylamides, vinyl, styryl, silicon oxide, boron oxide, phosphorous oxide, silicates, borates and phosphates.

A biological material is at least one of a: antibody, nucleic acid, polysaccharide, protein, drug, monoclonal antibody, antigen, polyclonal antibody, and other such biological material. Furthermore, a biological target at least one of a cell, protein, polysaccharide, drug, monoclonal antibody, polyclonal antibody antigen receptor, biological marker, peptide, protein. In an instance, the cartridge component 110 stores nanomaterial labels paired to biological materials in N wells, wherein N is an integer. The cartridge component 110 can be any solid support comprising rows of holes bored into the solid support to a specific depth, so as not to breach one side of the solid support. Each hole is known as a well and as such each well stores a nanomaterial label paired to biological materials. For instance, if N=100, there are 100 wells in the solid support is stored nanomaterial labels paired to biological materials.

The wells can be arranged in any order, such as ten rows of wells with ten wells in each row or even five rows of wells with twenty wells in each row. The wells can be organized in a symmetrical fashion or in no random order. An example of a cartridge component 110 is a microwell array, which can comprise over one million wells in some instances. Each well can act as an individual test environment whereby numerous simultaneous reactions can take place simultaneously.

In an embodiment, the floor of each well hole can hold mobile or immobilized biological materials (e.g. antibody, small molecule, liposome, protein, aptamer, toxin, peptide, growth factor, etc.). The biological materials can then be paired to a nanomaterial label (e.g. tetrapod quantum dot) by bio-conjugation, functionalization or bonding (e.g. coating the quantum dot with hydrophobic ligands which are linked to carboxylic acid) or bond (e.g. covalent, ionic, or hydrogen bonding, Van der Waals' forces, magnetics, or mechanical bonding) which are all well known in the art. For instance, in order to detect glucose in a subject's blood sample a well floor can hold immobilized glucose oxidase (“GOD”) (in an instance the GOD can be freely mobile too) whereby the GOD is paired to a CdSe tetrapod quantum dot. In another instance, a second well can hold a tetrapod quantum dot encapsulated with a sodium selective polymer matrix (e.g. sodium ionophore X conjugated with chromoinophore), which can change color based on the exchange of sodium ions (e.g. from sodium present in the biological sample, such as blood) that contact the sodium selective polymer matrix tetrapod quantum dot. Each well can store a different biological material paired to a unique nanomaterial label to perform a different test (e.g. glucose detection, sodium detection, etc.) whereby even all tests in a comprehensive metabolic panel can be performed simultaneously.

A comprehensive metabolic panel is a group of chemical tests performed on a subject, often using the subject's blood. In an aspect, the point of care device 100 can test for levels of albumin, alkaline phosphates (ALP), alanine aminotransferase (ALT), aspartate aminotransferase (AST), blood urea nitrogen (BUN), calcium, chloride, bicarbonate, magnesium, carbon dioxide (CO2), creatinine, glucose, potassium, sodium, total bilirubin, total protein, amylase, bilirubin, calcium, cholesterol, HDL cholesterol, LDL cholesterol, Cortisol, creatine kinase, creatanine kinase, estriol, ferritin, follicle-stimulating hormone, pH gas, PCo2 gas, Po2 gas, growth hormone-arginine stimulation, immunoglobulins, IgA, IgE, IgG, IgM, Iron, Lactate dehydrogenase, Luteinizing hormone, osmolality, parathyroid hormone, phosphatase (alkaline), Prolactin (hPRL), albumin, globulin, thyroid-stimulating hormone, thyroidal iodine uptake, thyroxine, triglycerides, triiodothyronine, triiodothyronine resin uptake, urea nitrogen, ureic acid.

Furthermore, in an aspect, the point of care device 100 also can test for hematologic indicators including but not limited to Bleeding time, CD4+ T-lymphocyte count, erythrocyte, erythrocyte sedimentation rate (Westergreen), Hematocrit, Hemoglobin A1C, Hemoglobin-blood, Hemoglobin-plasma, Leukocyte count and differential, leukocyte count, neutrophils-segmented, neutrophils-banded, eosinophils, basophils, lymphocytes, monocytes, mean corpuscular hemoglobin (MCH), Mean corpuscular hemoglobin concentration (MCHC), mean corpuscular volume (MCV), partial thromboplastin time, platelet count, prothrombin time, reticulocyte count, thrombin time, volume, plasma, red cell.

Additionally, in an aspect, the point of care device 100 can also test for cerebrospinal indicators including but not limited to cell count, chloride, gamma globulin, glucose, pressure, proteins. This device may also test for cerebrospinal indicators including but not limited to chloride, urine, calcium, chloride, creatinine clearance, estriol total, 17-hydroxycorticosteroids, 17-Ketosteroids total, osmolality, oxalate, potassium, proteins total, sodium, uric acid, body mass index (BMI). This device may also test for sexually transmitted diseases including but not limited to chlamydia, gonorrhea, herpes I, herpes II, hepatitis A, hepatitis B, hepatitis C, syphilis, HIV-1, HIV-II, human papilloma virus. In an aspect, the point of care device 100 can also test for other bacterial and viral infectious diseases including, but not limited to, influenza, Ebola, hantavirus, or staph aureus.

Additionally, in an aspect, the point of care device 100 can also test for cerebrospinal indicators including but not limited to anthrax, plague, small pox, malaria, influenza. This device may detect urinary tract infections including but not limited to Escherichia coli, straphylococcus saprophyticus, klebsiella, enterococci, ureaplasma urealyticum, mycoplasma hominis, proteus mirabilis. This device may also test for pyelonephritis, klebsiella, P. mirabilis, citrobacter, candida albicans, pseudomonas, aeruginosa, enterobacter, serratia, gram-positive organisms and S. saprophyticus.

In an aspect, a dispenser component 120 dispenses a biological sample into each respective well. The dispenser component 120 is a containment object, which stores a biological sample of a subject. The dispenser component 120 interlocks with the cartridge component 110 whereby the dispenser component can equally dispense and distribute the stored biological sample into each respective well of the cartridge component 110. In an aspect, the dispenser component 120 has one or more valves capable of opening and closing to dispense the biological sample into the wells. Each valve can be controlled individually or in unity to open or close. For instance, the valve associated with well 1 can be opened to distribute a sample of blood into the well whereas the valve associated with well 2 can remain closed so as not to dispense any blood into well 2. Furthermore, in an aspect, dispenser component 120 can comprise an injection opening whereby a biological sample can be injected into the containment chamber of the dispenser component 120 wherein the biological samples is stored until dispensed.

In an aspect, excitation component 130 emits energy from an excitation source to excite the nanomaterial label in the absence of the biological sample and excites the nanomaterial label in the presence of the biological sample. An excitation source is an energy source from which releases energy directed at the nanomaterial label for absorption. The nanomaterial label can be excited over a broad bandwidth, yet emits energy in a narrow band, which is uniquely identifiable by a detection component 140. The excitation component 130 can comprise an excitation source that emits electromagnetic radiation over a range of wavelengths such as x-ray, ultraviolet, visible, infrared, and any waves within that spectrum range. In another embodiment, excitation component 130 can comprise an excitation source that is a particle beam, such as an electron beam.

Nanomaterial labels are capable of excitation from an excitation source that emits electromagnetic radiation of a broad bandwidth allowing for simultaneous excitation of all nanomaterial labels in all the cartridge component 110 wells thereby enabling a single excitation source to excite several unique nanomaterial labels at the same time. Although, the excitation source emits at the same broad bandwidth, each unique nanomaterial label in each respective well will emit electromagnetic radiation at different frequencies, thus the detection component 140 is capable of detecting several different nanomaterial labels associated with the presence of several biological targets.

For instance, if well 1 stores glucose oxidase and a multi-leg luminescent nanomaterial comprising two legs paired to GOD and well 2 stores a multi-leg luminescent nanomaterial comprising three legs encapsulated by a sodium selective polymer matrix, both well 1 and well 2 will be exposed to the same electromagnetic radiation frequency but well 1 can emit radiation at a different frequency as the frequency of radiation emitted from well 2. Furthermore, detection component 140 can detect the change in the emission intensity exhibited from the two leg multi-leg luminescent nanomaterial in well 1 and the change in emission intensity from the three-leg multi-leg luminescent nanomaterial in well 2 in order to detect glucose in well 1 and sodium in well 2 from the same biological sample. In another aspect, the excitation component 130 emits energy from an excitation source to excite the nanomaterial label in the absence of the biological sample, that is, prior to the introduction of biological sample such as blood to the wells.

The emission wavelength of the nanomaterial label detected by the detection component 140 in the absence of the biological sample serves as a first emission metric detection component captures. The emission wavelength of the nanomaterial label detected by the detection component 140 in the presence of the biological sample and thus the presence of a biological target serves as a second emission metric detection component captures. The change in emission of radiation from the nanomaterial label between the first emission metric and the second emission metric is detected by the detection component 140 and further analysis component 150 analyzes the difference in metrics to determine the presence of a biological target (e.g. glucose, sodium) and the levels of concentrations of the biological targets present in the biological sample (e.g. blood, serum, urine, saliva, etc.). In an aspect, excitation component 140 can excite numerous (e.g. hundreds) nanomaterial labels simultaneously in order for detection component to detect numerous (e.g. hundreds) of biological targets simultaneously.

In an aspect, a detection component 140 detects a relative change in energy emitted from the nanomaterial label paired to the biological material in the absence of the biological sample as compared to the energy emitted from the nanomaterial label paired to a biological material in the presence of a biological target present in the biological sample. The detection component 140 is a tool that can capture electromagnetic emissions. In an aspect, detection component 140 can detect electromagnetic radiation or light (e.g visible, infrared, ultraviolet, x-ray, gamma ray, etc.) by utilizing the photoelectric effect, whereby light is absorbed in discrete packets called photons. Furthermore, light can be absorbed or emitted only as particles with discrete chunks of energy called quanta or quantum in the plural. When light is absorbed by the nanomaterial label, for instance a quantum dot, it interacts with the quantum dots electrons causing the electrons to transition between energy states. The light is absorbed in photons packets, whereby the photons (emitted from excitation source 130) “excite” the quantum dots electrons to higher energies. The energy of each photon is determined by its wavelength (but not its intensity).

When the quantum dots absorb the photons, the quantum dots electron increases in energy by the energy of the photon and when the quantum dot emits light, the electron's energy decreases by the photon's energy. The wavelength of the photon emitted from the quantum dot in the absence of a biological target (present in a biological substance) is different from the wavelength of the photon emitted by the quantum dot in the presence of a biological target. Moreover, this change in wavelength emitted in the absence of a biological target and in the presence of a biological target is detected by the detection component 140. Furthermore, the wavelength emitted by the quantum dot in the presence of a biological target will differ based on the level or concentration of biological target present in the biological sample. For instance, if there is more glucose present in a subject's blood, the wavelength emitted by a quantum dot in the presence of more glucose is different than the wavelength emitted by the quantum dot in the presence of less glucose, which is a detectable change detection component 140 can capture.

The detection component 140 can be any of a variety of detectors that quantify the change in wavelength emissions of quantum dots, such as a spectrometer for instance. In an embodiment, the detection component 140 can measure the absorption (or the emission) of light by a quantum dot. In another embodiment the detection component 140 can measure the time-varying intensity of light, which compares the intensity of light emitted or absorbed in some cases (y-axis) vs. wavelength (x-axis) to determine the fraction of light emitted (or absorbed in the case of absorption measurement) or what is commonly referred to as an emission (absorption in some cases) spectra.

Furthermore, the dilution of the biological sample can effect the quanta of light absorbed by the quantum dots in the presence of a respective dilution. For instance, if excitation component emits an initial intensity of light at the quantum dot in the presence of a dilute sample of blood, the amount of light absorbed by the quantum dot depends on the number of molecules of a particular biological target in the blood. For a very dilute solution (e.g. low amounts of glucose), the amount of light absorbed by the quantum dot is proportional to the concentration of glucose in the blood, the length of the light's path in the blood (whereby the concentration and length of light's path define how many molecules, of glucose for example, the light encounters) and a constant of proportionality known as an extinction coefficient which describes the quantum physics of the nanomaterial labels (e.g quantum dots in this example). Thus detection component 140 can capture data related to the biological sample that can determine the concentration of a biological target in a sample. However, analysis component 150 can analyze and make sense of the data captured by detection component 140.

In an aspect, analysis component 150 analyzes data related to the change in energy emitted from the nanomaterial label paired to the biological material as compared to a standard curve for the energy emitted from the nanomaterial label paired to a the biological material in the presence of various concentration levels of the biological target. In an aspect, analysis component 150 can analyze the data associated with the change in emission from nanomaterial labels captured by detection component 150 in order to determine the presence of a biological target and the concentration of respective biological targets in a biological sample. In an aspect, analysis component 150 can make use of a standard curve to determine the presence and concentration of biological targets in a biological sample.

A standard curve is a plot of multiple samples with known measured properties, which can be compared, to unknown samples to determine measured properties of the unknown sample. The samples with known properties are the standards (and the graph is the standard curve) by which the properties of an unknown sample are compared to by analysis component 150. For instance, analysis component 150 can compare a standard curve showing the absorbance of different concentrations of protein (e.g. milligrams of protein on the x-axis versus emission (or absorbance) wavelength of a quantum dot (which can also be noted as the absorbance wavelength by the detection component 140) in the presence of respective milligrams of protein on the y-axis) to emission wavelength data from a quantum dot (with the same properties as that quantum dot used to create the standard curve) in the presence of the respective protein in a subjects unknown sample, such as blood.

Analysis component 150 can determine the concentration of protein in the subjects blood by comparing the absorbance data (or emission data in some cases, so long as the comparison is always consistent, that is comparing standard absorption data to unknown absorption data; or standard emission data to unknown emission data) for the quantum dot in the presence of the subjects blood to absorption data for a quantum dot in a standard protein concentration. Thus, analysis component 150 analyzes data related to the change in energy emitted from the nanomaterial label paired to the biological material as compared to a standard curve for the energy emitted from the nanomaterial label paired to a the biological material in the presence of various concentration levels of the biological target. In an embodiment, the analysis component 120 can employ software to compute the concentration of one or more biological targets in a biological sample.

In an aspect, generation component 160 generates a medical diagnostic report based in part on the analyzed data. In an aspect, generation component can interpret the data analyzed by analysis component 150 and interpret such information into medically relevant information, such as the actual amount of concentration of a biological target in a subjects biological sample and output the concentration levels, the presence of the biological target, and provide medical feedback such as whether the concentrations warrant physician intervention, follow-up tests, potential diagnosis, and other relevant medical information. In an aspect, a subject is any human or animal.

In another aspect, biological sample can include blood, artery scarping, a blood clot, bodily fluids, serum, plasma, urine, vaginal fluid, mucus, lymph, blood byproducts, semen, saliva, sputum, spinal fluid, lymph fluid, skin, respiratory, intestinal, and genitourinary tracts, tears, milk, blood cells, tumors, organs, ocular lens fluid, amniotic fluid, in vivo cell culture constituents and also samples of in vitro cell culture constituents.

Display component 170 displays the medical diagnostic report to a device user. In an aspect, the display component 170 presents the data from generation component 160 to a device user. The display component 170 can be a touch screen, interactive monitor, LCD screen, projector screen, or any type of mechanism for presenting information. The display component 170 can display charts, graphs, text, words, numbers, numerous colors, and other such features. Additionally, display component 170 can comprise an audio feature whereby the medical diagnostic report can be audibly communicated to a device user and include alarm functions as well as audio sounds associated with monitoring needs (such as a sound related to low levels of glucose present in a subjects blood).

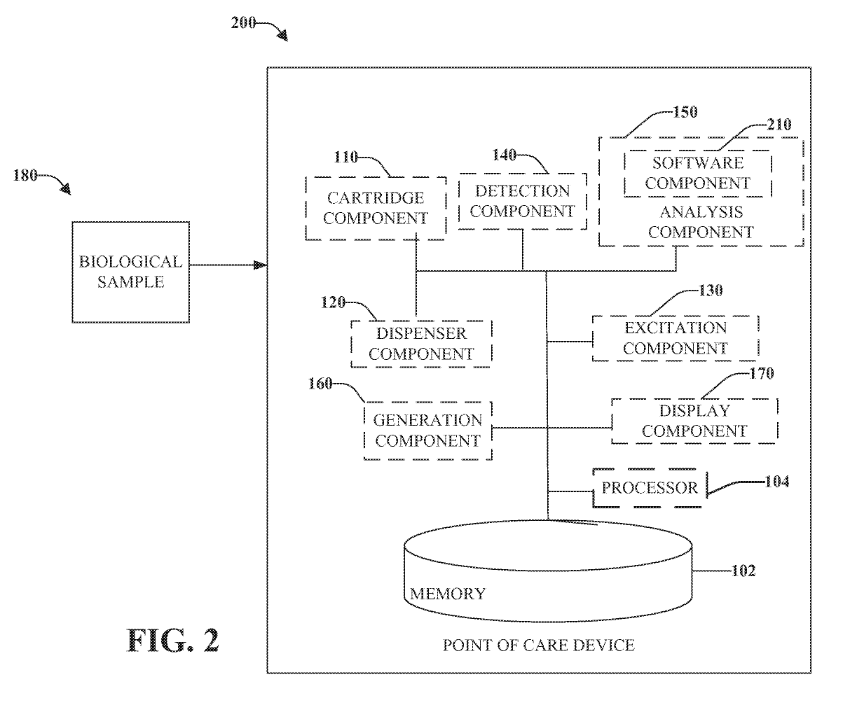

Turning now to FIG. 2, presented is another non-limiting embodiment of point of care device 200. In an aspect, analysis component 150 employs software component 210 that computes the concentration of one or more biological targets in a biological sample. In an aspect, the software component 210 can employ any analysis software that cart assess data from all other components of point of care device 200. For instance, software component 210 can analyze emission and absorbance luminescence data from the nanomaterial labels. Furthermore, in an aspect, software component 210 can analyze emission data from excitation component 130. In another instance, the software component 210 can analyze the data related to the amount of biological targets in a given biological sample and so forth. The software can be versatile and a variety of software's can be incorporated into the point of care device.

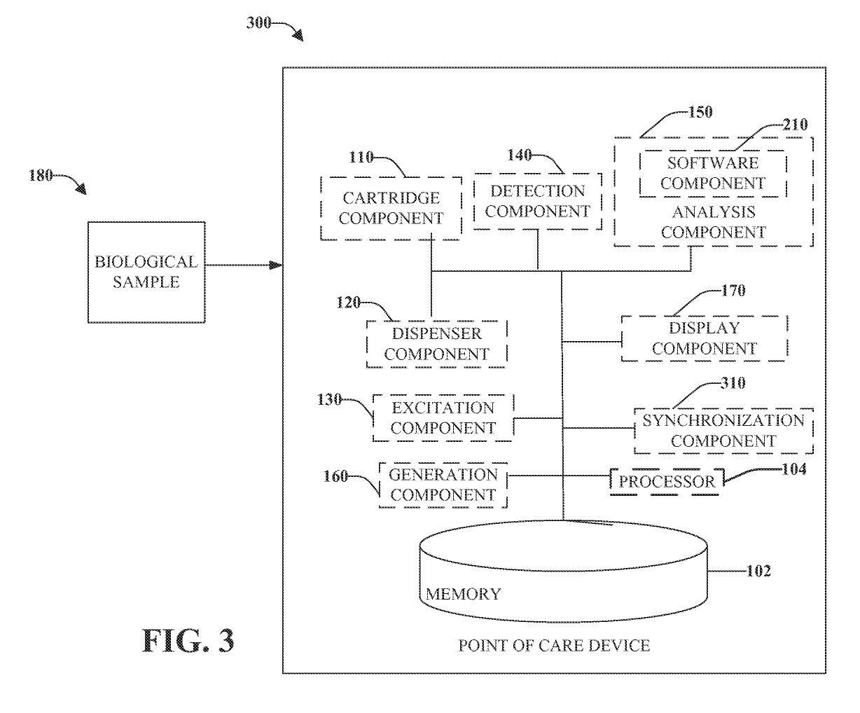

Turning now to FIG. 3, presented is another non-limiting embodiment of point of care device 300. In an embodiment synchronization component 210 synchronizes the device to a network system. The point of care device 200 is capable of integrating with other user devices such as laptops, tablets, desktop computers, mobile devices, personal digital assistants, and other such devices. As such synchronization component 310 gives the device the capability to make files on other devices. For instance, if a device user seeks to share the medical diagnostic report with other physicians or the subject, he can simply synchronize the device with other devices. Furthermore, a physician can synchronize the device with a network system such as a hospital electronic medical record system for fast, easy, digital transfer of the medical diagnostic report into a digital file associated with the client for the hospitals records. The patient can synchronize the device with his mobile phone or digital media player for easy, fast, and practical access to the users medical records in a convenient manner. Furthermore, point of care device 300 can be configured to operate on any of a mobile device, tablet computer, laptop computer, desktop computer, digital pen, medical testing tool or electronic device. The point of care device 200 can be built into mobile devices such as mobile phones for consolidation of all information on one single device.

In another aspect, the point of care device 300 can centrally store medical records electronically on a shared database. The database is capable of remote access by patients and medical providers. The system then stores numerous medical records on a medical information database via a medical information server connected to a network. A plurality of medical provider computers can be connected to the network and utilize software to communicate with the medical information server. Furthermore, the device can be configured to store other information such as medical history, surgical history, hospitalization, pregnancy history, medical treatment, gynecological history, pap history, allergy treatment, immunization treatment, family history, birth control history, vital sign and statistics, patient information, or physical exam notes.

In an embodiment, point of care device 300 can be utilized for glucose estimation in a subject's biological sample, which can be useful in determining conditions such as diabetes mellitus. In an embodiment, point of care device 100 utilizes quenching of fluorescence or emission spectrum's of quantum dots to determine the concentration of glucose present in a biological sample. The concentration of hydrogen peroxide (H2O2) detected in the biological sample by detection component 140 is associated with the concentration of glucose in the biological sample, wherein 1 mole of H2O2 is released from oxidation of 1 mole of glucose. A typical range of blood glucose levels from a hypoglycemic state to hyperglycemic state lies from 30 mg/dl (1.66 mM) to 360 mg/dl (20 mM). Nine quantum dot-ligand quenching systems were tested in an experiment was tested against samples along the typical blood glucose range. Each system was allowed 5 minutes of reaction time to present the sensitivity of each system in regards to glucose sensing via fluorescence quenching detection. The result was an increased quenching of the quantum dots emission wavelength with increasing concentrations of H2O2 quantified by the slope of relative fluorescence intensity (I/I0) versus H2O2 concentration, which is referred to as sensitivity.

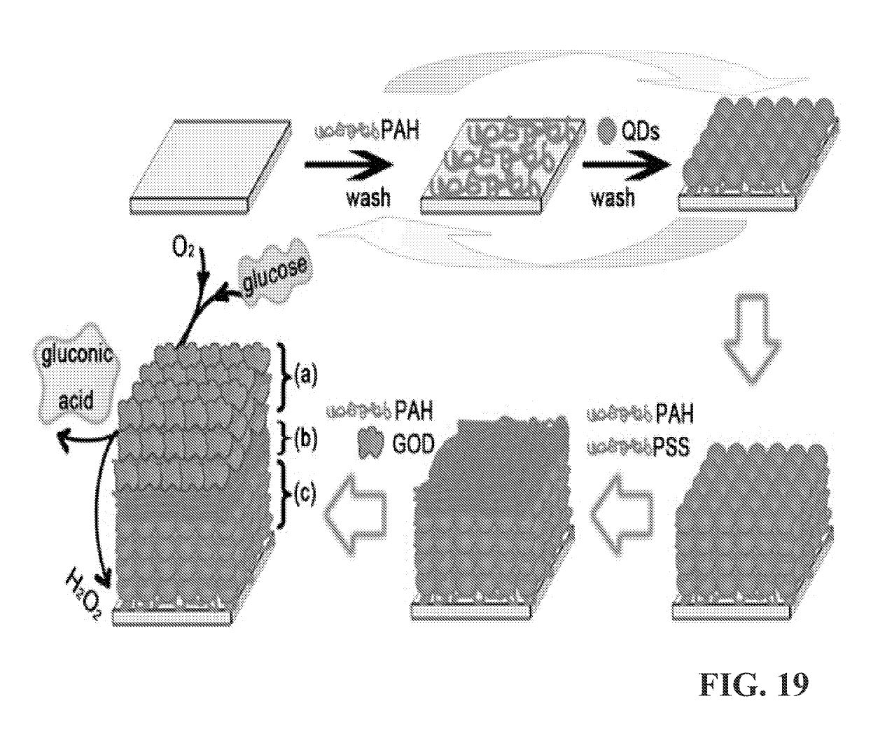

The experiment began with conjugating each water-soluble quantum dot-pairing moiety system (also known as quantum dot ligand system) to glucose oxidase (GOD). At the start of the experiment, the concentration of three spherical quantum dots; CdSe—CdS, CdSe—ZnS and CdS—ZnS were estimated as 0.32 mg/ml, 0.26 mg/ml and 0.23 mg/ml of PBS respectively, assuming complete recovery of the quantum dots during extraction from microemulsion into the PBS phase. Briefly, 2.5 mg of EDC (1 ml of PBS, pH=7.4) were added to 5 ml of each of the nine quantum dot-ligand systems (in PBS). The samples were then gently stirred for 30 minutes. Then, 2.5 mg of NHS (1 ml of PBS, pH=7.4) were added to all the samples and again stirred for 30 minutes. 1 ml of enzyme solution (1 mg/ml of GOD in PBS) was then added to each of the samples and stirred gently for 8 hours at 4° C. The quantum dot-ligand systems were then washed thrice and then redispersed in PBS. EDC coupled NHS to carboxyl groups on the quantum dots surface, resulting in formation of NHS esters, which reacted with the amine group of GOD, to form an amide linkage. The mechanism is clearly depicted in FIG. 19

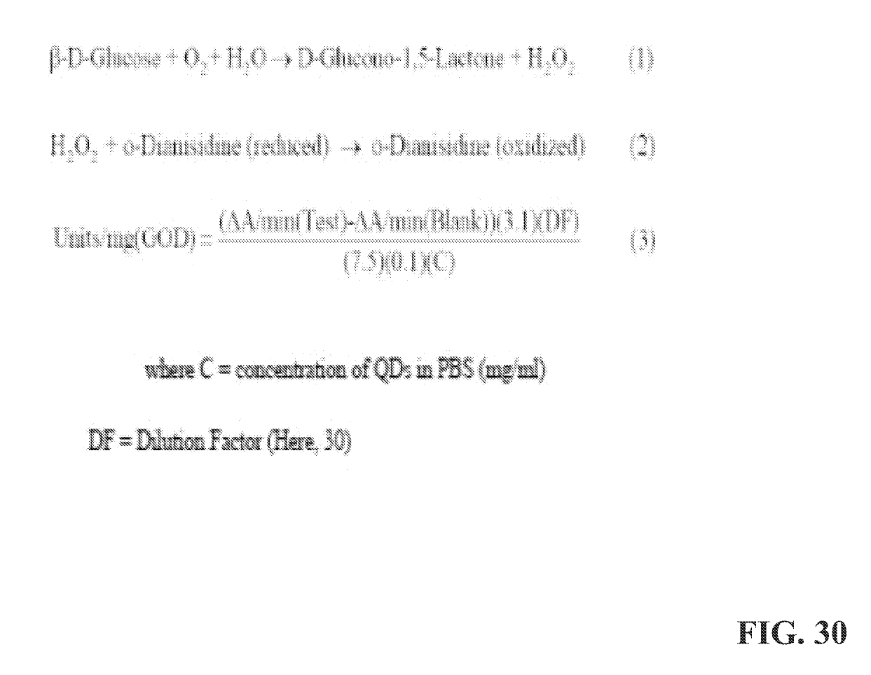

Next, the enzyme activity and stability of the quantum dot-ligand-GOD (QD-ligand-GOD) systems were observed. The estimation of Enzyme Activity (EA), which are the Units of GOD per mg of Quant Dots, was performed as per a method reported by Sigma-Aldrich (2010), for each of the nine QD-ligand-GOD systems. The mechanism of activity estimation is explained by equations 1 and 2 and activity is calculated as per the equation in FIG. 30 where C=concentration of QDs in PBS (mg/ml), DF=Dilution Factor (Here, 30). The enzyme activity was obtained for 0, 2 and 4 weeks, thus giving the stability of the GOD conjugated on the QD surface. Quantification of this stability is represented here by a parameter, Enzyme Stability Coefficient (ESC), defined as percentage of activity retained by the QD-ligand-GOD system after keeping the samples at room temperature for four weeks.

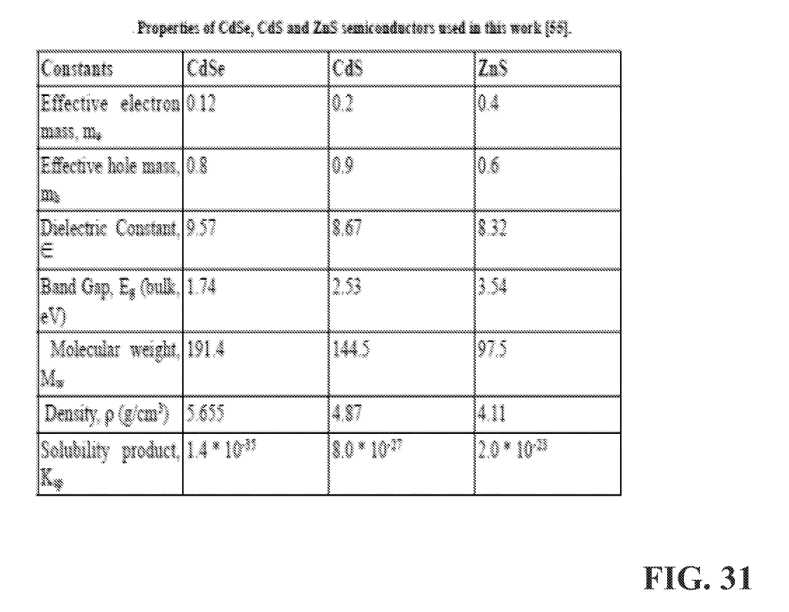

During characterization of the QD-ligand systems, the systems were analyzed for particle size, shape and crystallographic information in addition to the quenching effects the presence of H2O2 in the measured sample had on quantum dot fluorescence. The absorbance spectra for the nine QD-ligand systems in PBS were determined using UV-visible spectrophotometer (Nicolet Evolution 300). The band gap of QDs were determined by fitting the absorbance data to the following Equation: (σhν)2=A (hν−Eg) where, σ is molar absorption coefficient, A the proportionality and hν the photon energy. The mean size of core QD can be calculated by using the relationship between, QD band gap Eg (obtained from Equation 4), bulk band gap Eg (bulk), and mean particle diameter dp, which is expressed by Brus [54] as follows in the following Equation (A):

$${E}_{QD , d} = {E}_{bulk} + \frac{{h}^{2}}{2 {d}^{2}} ( \frac{1}{{m}_{e}^{*}} + \frac{1}{{m}_{h}^{*}} ) - ( \frac{3.6 {e}^{2} }{4 Π ɛ {ɛ}_{0} d} )$$

Where h is Planks constant, ∈ is dielectric constant of semiconductor, e the charge of electron, m*e=me.m0 and m*h=mh.m0. Here me and mh are the effective masses of electron and hole for core QD, and m0 is the mass of an electron. By using Equation (5), we estimate the mean sizes of nine QD-ligand systems. The universal constants used are m0=9.1*10-31 kg, ∈o=8.854*10-12 F/m, e=1.6*10-19 C, h=6.626*10-34 J/s. The individual constants for CdSe, CdS and ZnS are taken from the CRC handbook and complied in the table shown in FIG. 31. Regarding the nine core-shell QDs, while calculating the particle size from Equation (A), noted above, the core-shell was assumed to be entirely core material. This is in accordance to an analysis done by Loukanov et al. as per which, the shell contribution to the exciton energy can be ignored.

Further, the fluorescence spectra of QD-ligand and QD-ligand-GOD systems were obtained from Hitachi Fluorescence Spectrophotometer F-2500. The slit width was fixed at 10 nm for both excitation and emission windows while the excitation wavelength was kept at 420 nm.

The micrographs of the QD-ligand system were obtained with Philips Technai G2 at 120 kV. Additionally, High-Resolution Transmission Electron Microscopy (HRTEM) micrographs and the Electron Diffraction (ED) patterns were obtained from JEOL FEG-TEM at 200 kV. A drop of QD-ligand buffer dispersion was directly placed on a copper grid and dried overnight before the analysis was performed.

The experimental materials used in the synthesis, characterization, and selection of the quantum dot-ligand system included the surfactant, dioctyl sulfosuccinate sodium salt (Aerosol-OT, or AOT, 99% pure), Cadmium nitrate (LR grade), Zinc nitrate (LR grade), n-heptane (extrapure, 99%) were purchased from S.D. Fine (India) chemicals. Ammonium sulfide ((NH4)2S) (25% aqueous), Mercaptoacetic acid (97%, aqueous), Mercaptopropionic acid (99%, aqueous) and Sodium Sulfite (anhydrous) were purchased from Alfa Aesar, (U.K). N-Hydroxysuccinimide (98%) and L-Glutathione (reduced 98%) was obtained from Aldrich. Selenium powder (black, GR grade) of 99.5% purity was supplied by Kemie Labs, India. D-Glucose (minimum 99.5%), Glucose Oxidase (G2133 type VII from Aspergillus niger, 160,000 U/g solid), Peroxidase (P8375, Type VI from Horseradish, 250 Purpurogallin units/mg), Phosphate Buffered Saline tablets (P4417) and o-Dianisidine (D9143) were obtained from Sigma. N-(3-Dimethylaminopropyl)-N′-ethyl-carbodiimide hydrochloride (98% (AT)) was obtained from Sigma-Aldrich. Hydrogen peroxide (50% aqueous solution) was obtained from Qualigens Fine Chemicals. All the chemicals were used without any further purification. Ultra-pure water (Milli-Q, Millipore) was used throughout the experiments.

The microemulsion synthesis of CdSe—CdS, CdSe—ZnS and CdS—ZnS coreshell QDs and their extraction in aqueous phase was performed as per an ecofriendly method recently reported by Saran and Bellare. Briefly the core-shell QDs were prepared using AOT\water\n-heptane microemulsion system at room temperature and then extracted using appropriate thiol ligand into PBS buffer as per a Post-Synthesis Stabilization (PostSS) method. Each core-shell QD was extracted using 1 M aqueous solutions of MAA and MPA and 0.5 M aqueous solution of GSH. The core-shell QDs were synthesized at water-to-surfactant molar ratio (R=10) and shell-to-core molar ratio (S=2). Finally 10 ml microemulsion of core-shell QDs was extracted to 5 ml of PBS, ultrasonicated for 60 seconds and washed three times.

Experimental Results and Discussion.

The microemulsion synthesis of ligand capped QD's offered an 80% recovery of heptane and 40% recovery of surfactant AOT. The QDs were functionalized with mercaptothiol ligands, detached from the organic phase and extracted into a stable aqueous buffer in a single step. Each of the three ligands MAA, MPA and GSH were used to extract/cap each of the three QDs; CdSe—CdS, CdSe—ZnS and CdS—ZnS.

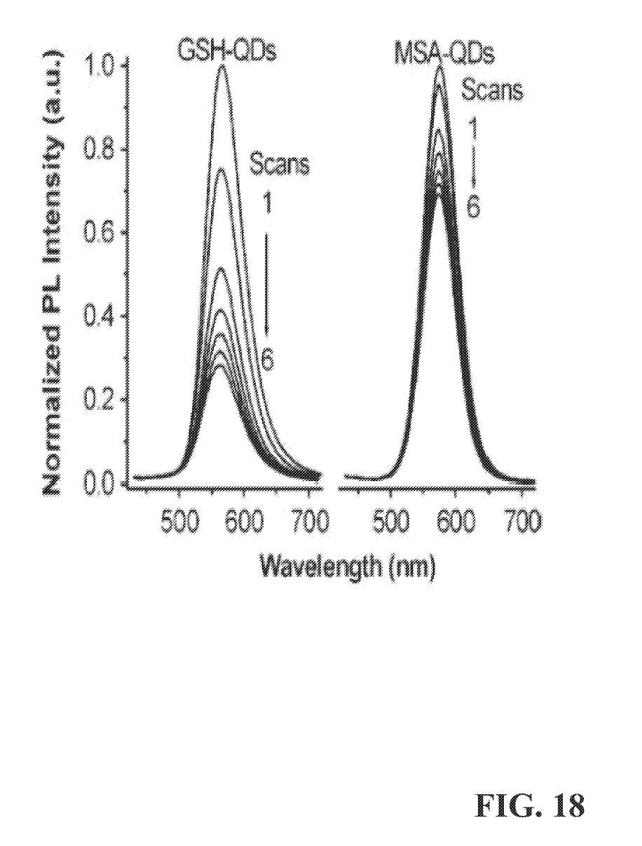

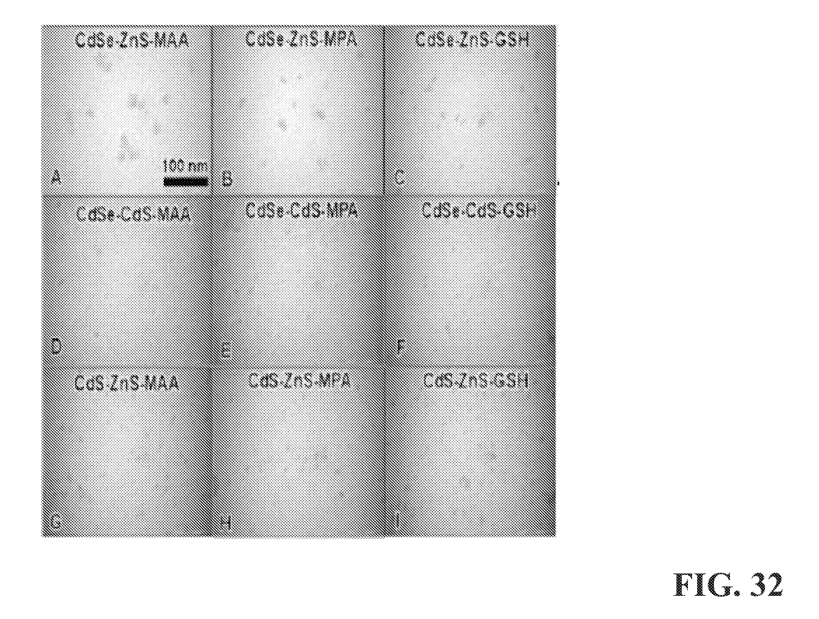

The nine QD-ligand systems were studied with TEM, HRTEM and ED to obtain information about nanoparticle size and shape as well as crystallographic properties. FIG. 32 gives the TEM micrographs for the nine systems. FIG. 32 32 (A, B, C) are of CdSe—ZnS capped with MAA, MPA and GSH respectively. The nanoparticles appear roughly spherical and show no signs of agglomeration thus indicating high stability of the aqueous dispersions. The mean particle size of each system was estimated by averaging the size of about 20 particles. CdSe—ZnS shows a mean size of 6.52, 6.34 and 5.89 nm for MAA, MPA and GSH. A decreasing particle size with corresponding increased ligand size (MAA<MPA<GSH) was an expected outcome because larger ligands lower the level of aggregation (due to increased steric repulsion) and thus lower the mean size. Similar observations apply to CdSe—CdS (FIG. 32 D, E, F) and CdS—ZnS (FIG. 18 G, H, I). CdSe—CdS exhibits a mean size of 6.01, 5.83 and 5.52 nm and CdS—ZnS shows a mean size of 5.22, 5.10 and 4.76 nm for MAA, MPA and GSH respectively. CdS—ZnS QDs were the smallest followed by CdSe—CdS with the CdSe—ZnS as the largest (as explained by the molecular weight and bulk density data given in the Table shown in FIG. 31).

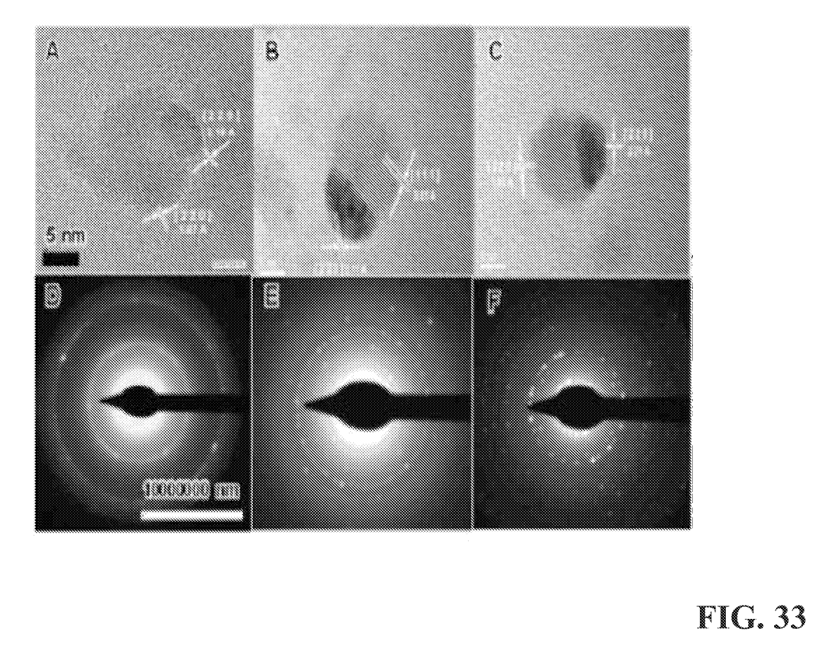

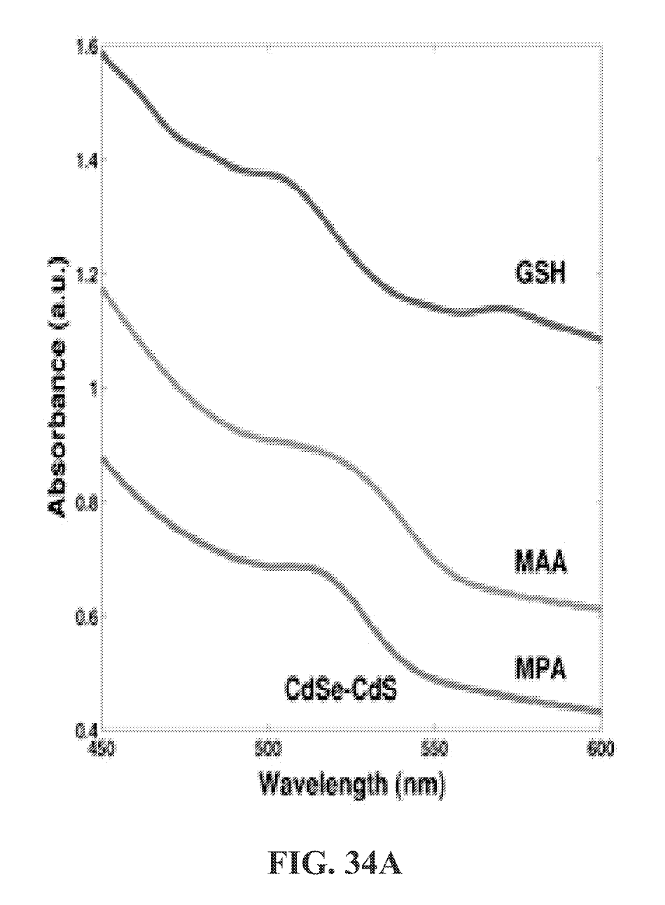

FIG. 33 shows HRTEM micrographs and ED patterns for CdSe—ZnS (A and D), CdSe—CdS (B and E) and CdS—ZnS (C and F). The HRTEM micrographs clearly show the lattice fringes for both core and shell material. The core was electronically heavier than the shell in all the three core-shell QDs. Consider FIG. 33(A) where lattice spacing of CdSe is calculated to be 2.19 Å that corresponds to plane (220) and the spacing of ZnS is found to be 1.87 Å corresponding to plane (220) of ZnS. In the case of CdSe—CdS (FIG. 33 (B)) the core spacing of 3.81 Å corresponds to plane (111) while the shell spacing is 2.11 Å corresponding to plane (220) of CdS. From FIG. 33(C) we estimate the core spacing as 2.21 Å corresponding to plane (220) of CdS and that of shell is 1.83 Å corresponding to plane (220) of ZnS. The selected-area electron diffraction patterns for the three QDs confirm the face centered cubic lattice system (zinc blende) (data taken from JCPDS file nos. 80-0019, 19-0191 and 77-2100).

In an embodiment, point of care device 100 can detect the presence and concentrations of electrolytes, potassium, calcium and bicarbonate utilizing nanomaterial labels. In Diabetes Ketoacidosis (DKA), the estimation of serum Na+, K+, HCO3- and pH are of utmost importance as acidosis leads to derangement of ionic physiology. Due to the increase of H+ ions an efflux of the K+ ions moves into the extracellular fluid, and accordingly an influx of H+ ions into the cells also results. This leads to cellular acidosis and hyperkalemia. Also, due to the acidosis there is depletion of the bicarbonate ions. Dehydration and hyperkalemia leads to hypernatremia.

In DKA, the pH of the blood ranges from 6.3 to 7.3. The potential clinical effects of hypernatremia, hyperkalemia and acidosis are dehydration, neural dysfunction, respiratory distress, respiratory failure, cellular death, thus it is important for a subject to estimate serum electrolytes and pH regularly. Currently, the conventional method of serum electrolyte and pH of estimation is through laboratory testing using colorimetric analysis, pH probes and certain amperometric analyzers. The major problem is the sample collection and the mode of transport. For serum electrolytes the sample has to be collected in plain blood and for pH arterial sample has to be taken and then transported in an ice bath. The transportation and laboratory reporting causes a delay in the appropriate treatment. Thus, the point of care device 100 as a detector for electrolytes and pH will provide tremendous benefits to the healthcare industry.

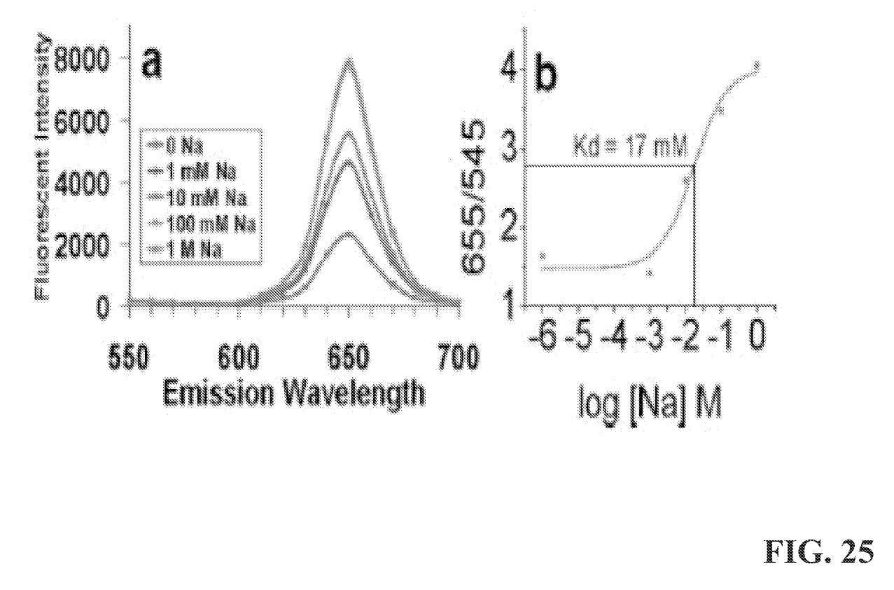

In an embodiment, point of care device 100 can be used to detect sodium levels in a biological sample. Dubach et al. (2007) reported the use of ion selective polymer and quantum dot for sensing sodium concentration. The ion-selective polymer is based on the traditional ion-selective optical sensors (optodes). Sodium ionophore X in conjugation with chromoionophore (a light absorbing pH indicator) formed a polymer matrix. This sodium selective polymer matrix was then used to encase quantum dots. The mechanism of sensing was based on the exchange of the sodium ions in the polymer matrix for the H+ ions thus decreasing the pH of the surrounding medium. This lead to a change in the chromoionophore color and thus indirectly determined sodium concentration. The absorption of the chromoionophore overlapped with the emission wavelengths of the encased QD. The assembly and the basis of FRET between QD and polymer are clearly shown in FIG. 25.

The results of the sodium selective polymer matrix encased quantum dot assembly are shown in FIG. 25(a). The fluorescence spectra clearly shows the increase in the emission of QD with the increase in sodium concentration, which can be calibrated ratiometrically as shown in FIG. 25(b). Point of care device 100 can utilize this technique within one well on cartridge component 100 to detect sodium levels in a biological sample. The sodium selective polymer matrix encased quantum dot can be stored in a well whereby upon presence of sodium, the energy emitted from the sodium selective polymer matrix encased quantum dot can be detected by detection component 140 and further analyzed by analysis component 150. Analysis component 150 can compare the sodium selective polymer matrix encased quantum dot emission wavelength to a standard curve for respective emission wavelengths related to standard concentrations of sodium in order to determine the concentration of sodium in the unknown blood sample.

In an embodiment, point of care device 100 can be used to detect urea in a biological sample. Urea is an important metabolite, which helps to determine the integrity of hepatic and renal physiology. A breach in the functioning of the liver or kidneys leads to the increase in the levels of urea (>60 mg %). Urea in blood is actually an indication of nitrogen levels in the blood. Usual range of urea is 7 mg % to 21 mg %. Urea is produced in the liver and is excreted by the kidneys. Thus increase in the production or the decrease in the excretion is an indication of hepatic or renal pathology, respectively. Usually the test to determine the urea levels is either a colorimetric or conductometric test. These laboratory tests are well established and require special blood sampling methods. The blood needs to be collected in an anticoagulant bulb for further evaluation, however, serum urea estimation can also be conducted. In an embodiment, point of care device 100 can be used to detect urea based on the science underlying the hydrolysis of urea into ammonia by enzyme urease. Thus, the analytical performance of these system is based on the stability of the enzyme, urease.

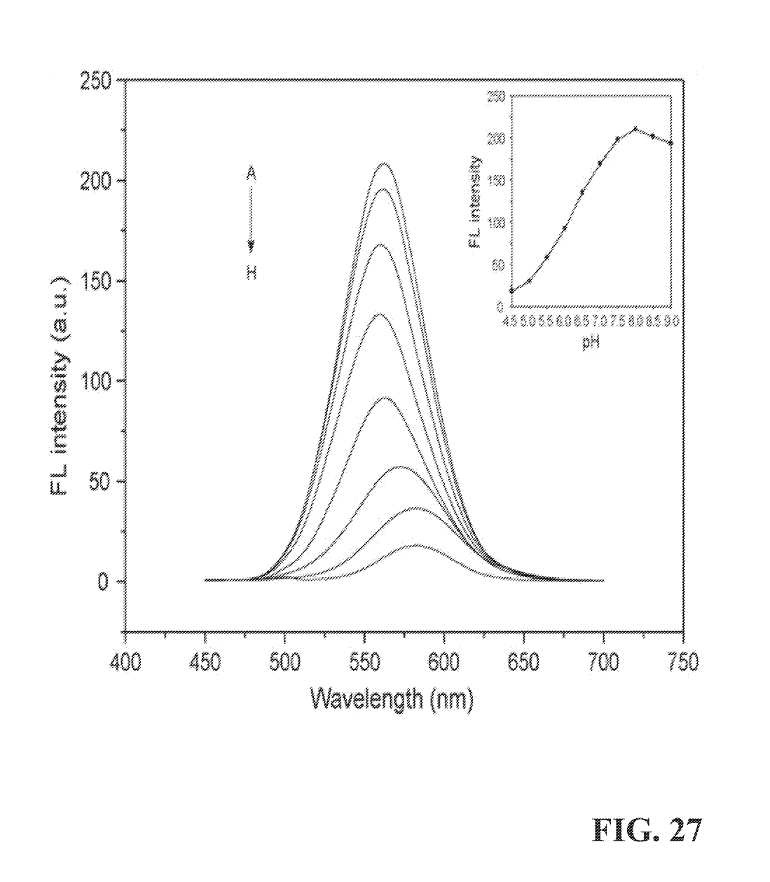

Huang C. P. et al. in 2006, reported the use of quantum dots for sensing urea. They used CdSe/ZnS quantum dots for the determination of urea, based on the phenomenon of fluorescence change with respect to the change in pH. When urea is hydrolyzed to ammonium ions with the help of a catalytic enzyme, urease, HCO3 ions and OH— are released and that causes the pH to increase. The results reported (FIG. 27) were promising and the increase in fluorescence was found to be linearly correlated with the increase in pH.

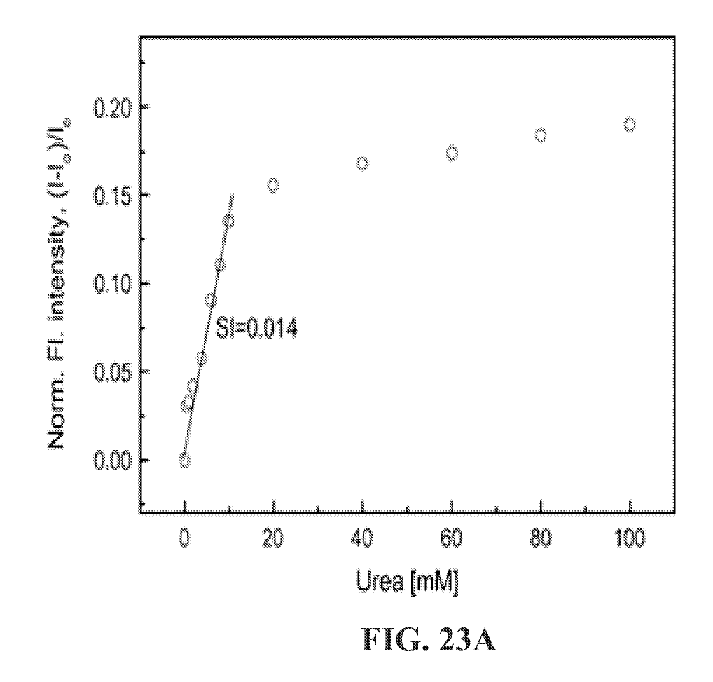

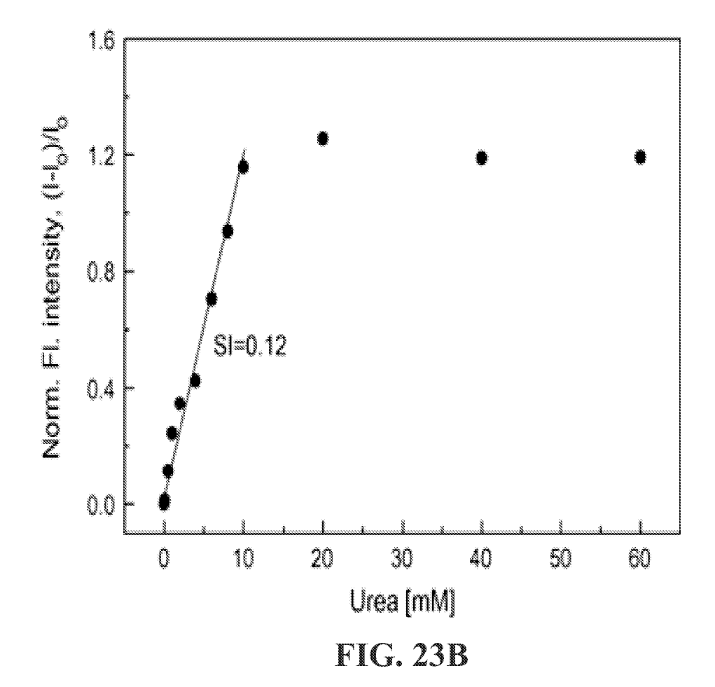

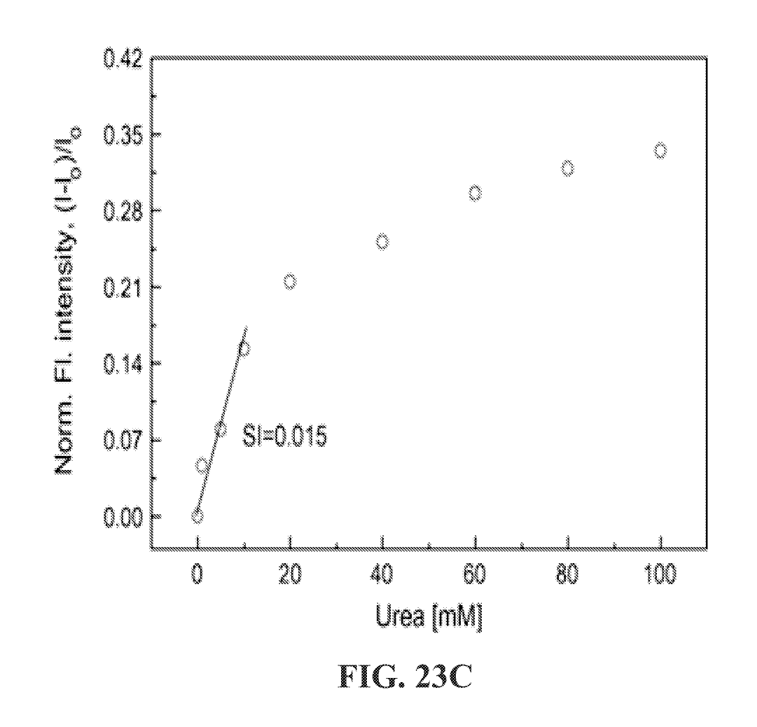

In 2008, Duong and Rhee demonstrated the application of quantum dots and sol-gel matrices in the estimation of urea. In the process they made three assemblies; QD-trapped sol-gel membranes, Urease-immobilized sol-gel membrane and Double layer consisting of QD-entrapped sol-gel membrane and Ureaseimmobilized sol-gel membrane. The quantum dots they used were mercaptopropionic acid capped CdSe—ZnS (CdSe—ZnS-MPA). The sol-gel membranes were made of 3-glycidoxytrimethoxysilane (GPTMS) and 3-aminotrimethoxysilane (APTMS). The result of the sensing ability by the three membranes is depicted in FIG. 23. The sensing was based on the change of pH with the hydrolysis of urea. The pH sensitivity of CdSe—ZnS QDs and the sol-gel immobilization technique were successfully used to fabricate urea sensor. The linear detection range of their model was 0-10 mM. They also studied the stability of the sol-gel membranes over two months with positive outcome with a conclusion of their aptness in biomedical and environmental applications.

In an embodiment, point of care device 100 can be used to detect urea in a biological sample by utilizing QD-trapped sol-gel membranes, Urease-immobilized sol-gel membrane and Double layer consisting of QD-entrapped sol-gel membrane and Urease immobilized sol-gel membrane techniques within respective wells of cartridge component 110. When a biological sample containing urea is dispersed within the respective wells, the urea can be hydrolyzed to ammonium ions with the help of a catalytic enzyme, urease. Upon the hydrolyzation of the urea into ammonium ions HCO3 ions and OH— are released and that causes the pH to increase. The detection component 140 can detect the change in wavelength emitted (via excitation of excitation component 130) from the quantum dot in the presence of the increased pH amounts. Furthermore, the analysis component 150 can compare the emission wavelength to a standard curve for emission wavelengths associated with standard concentrations of urea in order to determine the concentration of urea in the unknown sample. Generation component 160 can generate a medical diagnostic report based in part on the analyzed data and display component 170 can display the medical diagnostic report to a device user.

In an embodiment, point of care device 100 can be used to detect potassium, calcium, and bicarbonate in a biological sample. Singh et al. (2009) used a complex assembly to determine the concentration of potassium ions and calcium. They conjugated the Schiff base receptor to QD surface and demonstrated that the QD surface works as a scaffold for the organization of receptor enabling semi-selective binding to ions. The fluorescence response was found to be linear between 15-50 μM for potassium and 2-35 μM for calcium. They also demonstrated that the model could measure both potassium and calcium in solutions containing both ions. With the help of peroxide-bicarbonate system, the bicarbonate levels may be measurable. The mode of sensing can be the chemiluminescence resonance energy transfer between the peroxymonocarbonate ions and Quantum dots.

In an embodiment, point of care device 100 can be used to detect potassium, calcium, and bicarbonate. The detection component 140 can detect the change in wavelength emitted by the quantum dot in the absence of potassium and in the presence of potassium. Analysis component 150 can analyze the change in wavelength emission and compare the emission change to a standard curve of known standard concentrations of potassium. The comparison of the potassium levels from the unknown biological sample to the known standard sample can be analyzed by analysis component 150 to determine the concentration of potassium for the unknown biological sample. The device can perform the same process to detect calcium, bicarbonate in a biological sample. Each well can store a different biological material and perform a different detection test simultaneously.

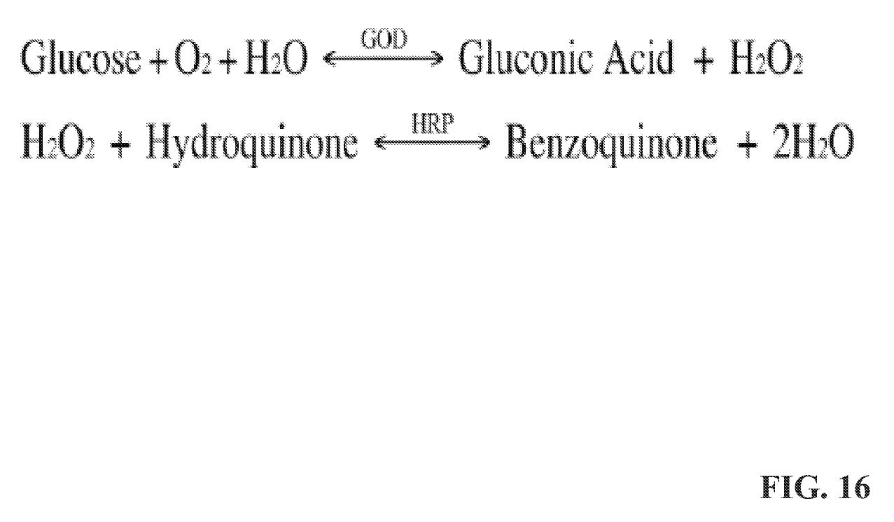

In an embodiment, point of care device 100 can be used to detect pH levels of a biological sample. The possible use of quantum dots in determining the pH of a solution has been well studied by the researchers around the globe. In 2006, a quantum dot based pH probe was assembled for the brisk study of the enzyme kinetics of various reactions. Yu D. et al., used CdTe—ZnS core-shell QDs to study the reaction kinetics of hydrolysis of glycidyl butyrate by the catalyst, procine pancreatic lipase (PPL). They also compared their methodology to the existing method based on p-Nitrophenoxide (PNP). The graph showing the variation of the fluorescence with respect to the solution pH is depicted in FIG. 16 They also showed the variation of temperature on the fluorescence of the QDs. The effect of ionic strength of the buffer was also well established in there work.

In 2007, a novel use of Quantum dots was floated. The use of QDs as a proton flux sensor and H9 influenza virus detector. Yun et al., used chromatophores from Rhodospirillium rubrum and labeled them with CdTe QDs stabilized by thiolglycolic acid. These QD-labeled chromatophores were then used to construct the QD virus detector. However, they too reported the variation of QD fluorescence with respect to pH change. The findings by the group are shown in FIG. 27 Maule et al. (2009) and Wu W. et al. (2010), reported Quantum dots as a probe for sensing pH [51,52].

In an embodiment, the point of care device can be used to detect pH levels in a biological sample. The change in fluorescence emission of the quantum dot in the presence biological sample in the absence and presence of pH can be detected by detection component 140. Analysis component 150 can analyze the change in wavelength emission and compare the emission change to a standard curve of standard levels of pH in a known sample. The comparison of the pH levels from the unknown biological sample to the known standard sample can be analyzed by analysis component 150 to determine the concentration of pH for the unknown biological sample.

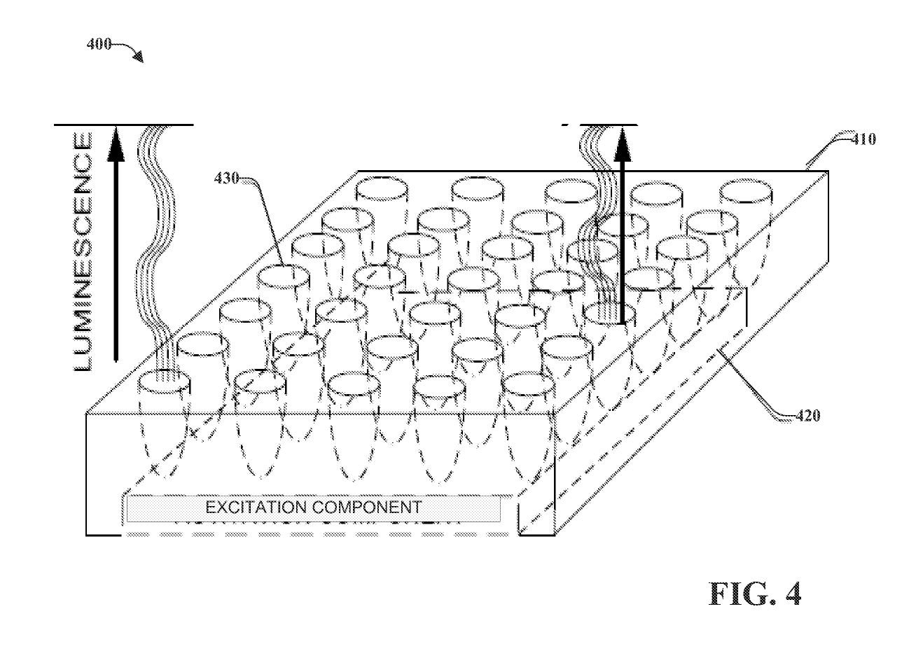

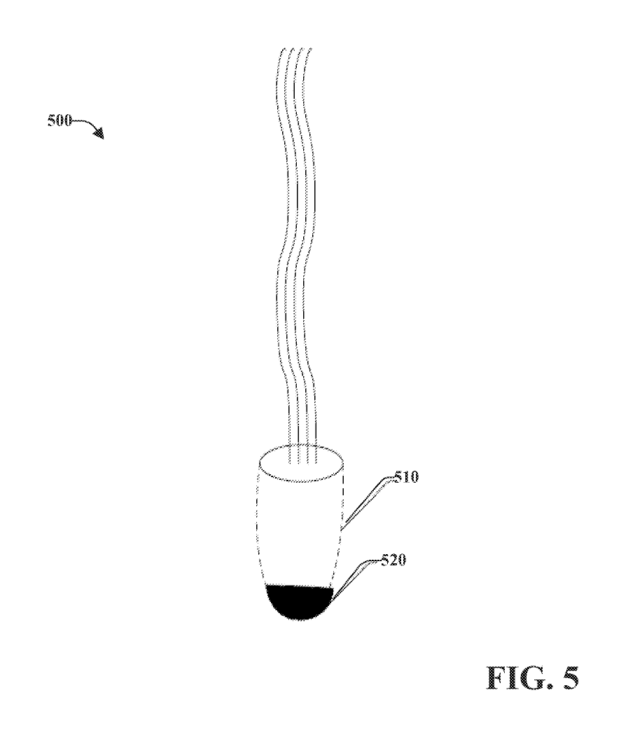

Turning, now to FIG. 4, disclosed is a non-limiting example of a cartridge component 400. In an aspect, cartridge component 400 comprises rows of individual well 430. The well 430 are embedded in a solid state tray 410 of a durable material (e.g. plastic, glass, steel, metal, etc.). At, 420 a lower layer of solid support can sit below the wells. In an aspect, the wells can even comprise an excitation component either above the top open portion of the well or below the bottom closed portion of the well at 420. Turning now to FIG. 5, disclosed is a non-limiting example of an individual well 500 of a cartridge component 400. The individual well 500 comprises at least one biological material and a nanomaterial label 520 stored in the well 500. The well itself has an adjustable depth 510 that can be varied depending on various factors such as the amount of biological sample to be tested in each individual well 500 or the amount of biological material and nanomaterial label 520 to be present in each well. Furthermore, luminescence from the nanomaterial label can be directed upward out of the individual well 500 as illustrated by the wavy lines depicted in the illustration. Furthermore, the excitation source can enter the individual well 510 through the top opening of the well or the bottom of the well (in the case that the well is constructed from a transparent or translucent material).

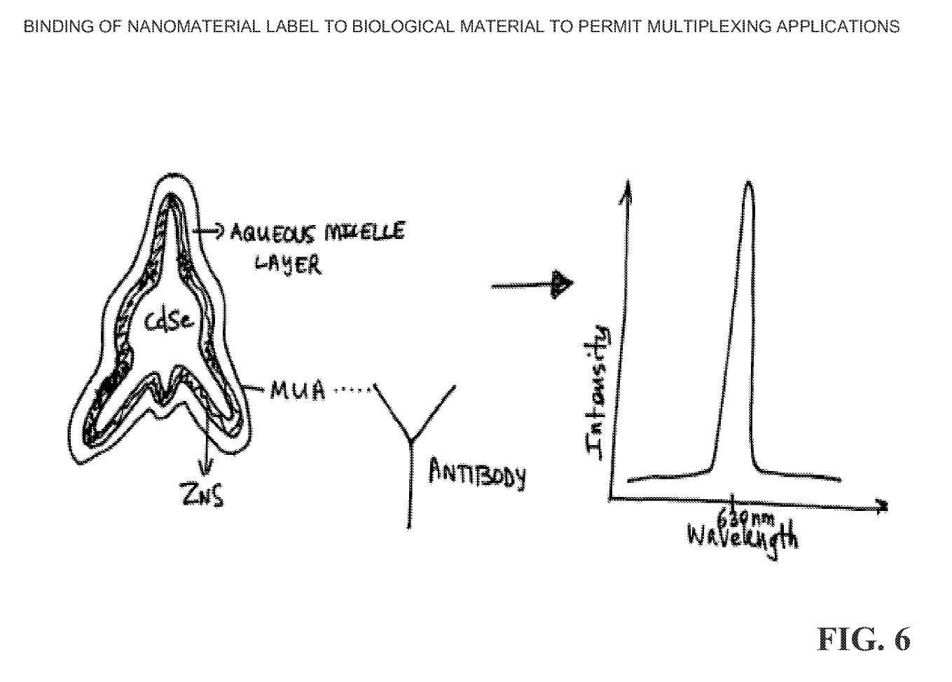

Referring now to the FIG. 6, a non-limiting example of a nanomaterial label bound to a biological material to permit multiplexing applications is shown. In an aspect, the nanomaterial label can be a multi-leg luminescent nanoparticle (MLN) with a CdSe base material and a ZnS shell material. Furthermore, the MLN can be encapsulated by an aqueous micelle layer around the ZnS shell. The MLN nanomaterial label can be paired to a biological material, such as an antibody, by a pairing moiety indicated by MUA in FIG. 6. Multiple MLN's each possessing a unique spectral signature can be used to detect a unique biological target in each individual well. Furthermore, each unique MLN or nanomaterial label can be read by a unique narrow wavelength band illustrated by the intensity on the y-axis and wavelength (of 630 nm in the example shown in the FIG. 6) on the x-axis.

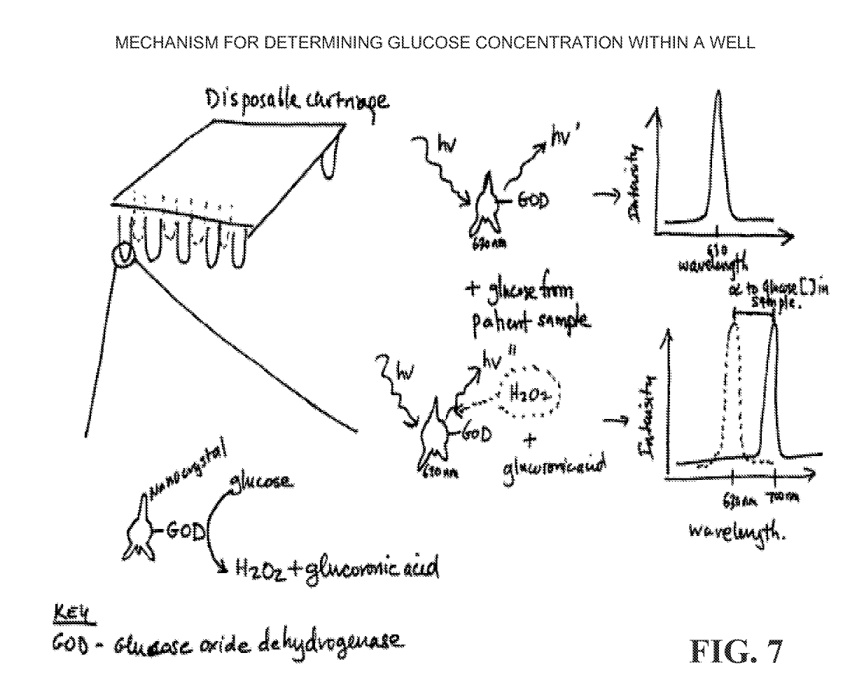

Referring now to FIG. 7, a non-limiting example of a nanomaterial label bound to a mechanism for determining glucose concentration within a well is shown. In an aspect, a disposable cartridge comprising multiple wells comprises an individual well storing a nanomaterial label, such as a tetrapod quantum dot paired to a glucose oxide dehydrogenase molecule (GOD). The nanomaterial label can be coated with inorganic capping agents and then pairing moieties, such as inorganic ligands, to protect the nanomaterial labels properties from an external environment that may curb its properties. The absorption and emission spectrum of the nanomaterial label paired to the GOD is captured by the detection component. Then a biological sample, such as patient blood is dispersed by dispersing component into the cartridge component and this into the individual wells whereby light is applied to the biological sample interspersed with the nanomaterial label and biological sample. If glucose is present in the patient blood sample, the glucose will GOD will cleave the glucose into hydrogen peroxide and gluconic acid. The hydrogen peroxide will quench the fluorescence of the nanomaterial label thereby producing a different emission wavelength by the nanomaterial label than prior to the addition of the biological sample. A comparison of the two wavelength's will indicate the concentration of glucose present in the blood sample. Thus, the FIG. 7 demonstrates the shift from a 630 nm wavelength to a 700 nm wavelength emission in the presence of glucose from a biological sample. This shift corresponds to a specific concentration of glucose as indicated by a standard curve.

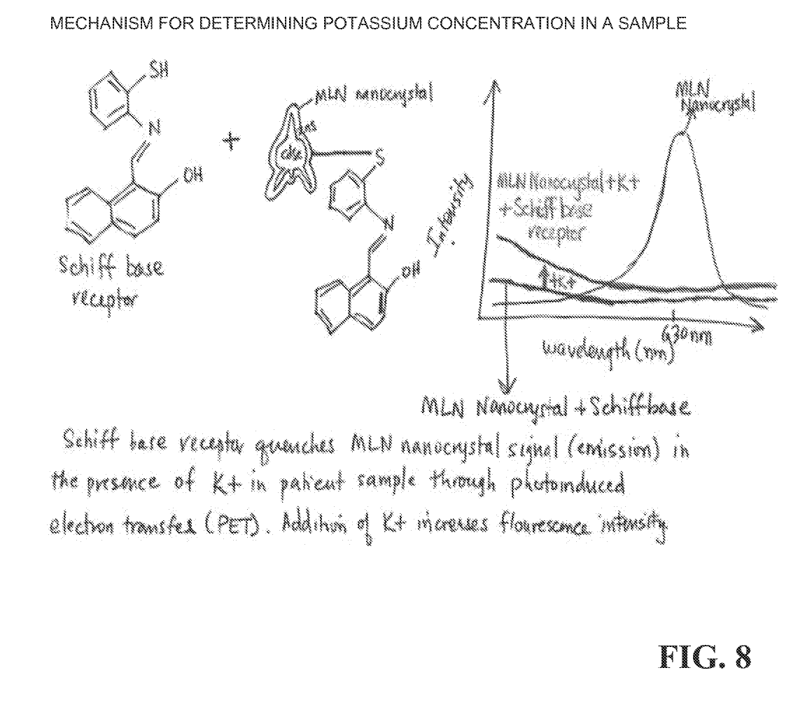

Referring now to FIG. 8, a non-limiting example of a mechanism for determining potassium concentration in a sample is shown. In an aspect, a Schiff base receptor is paired to a nanomaterial label, such as an MLN. The nanomaterial label and Schiff base receptor are stored in a well in a cartridge component. Upon the dispersion (e.g. using dispersing component) of a biological sample into the well storing the Schiff base receptor paired to the MLN, the presence of potassium in the biological sample quenches the MLN emission signal through photo-induced electron transfer (PET). The presence of potassium will increase the fluorescence intensity of the MLN or other nanomaterial label. The same can be performed with calcium ions using functionalized CdSe core and ZnS shelled quantum dots as demonstrated by Singh, et. Al. (2009); J. Fluorescence, 2009, 19; 777-782. The MLN nanocrystal prior to the addition of the biological sample as compared to after the addition of the biological sample comprising potassium will cause a shift in the wavelength emission spectrum which can be detected by detection component of the device.

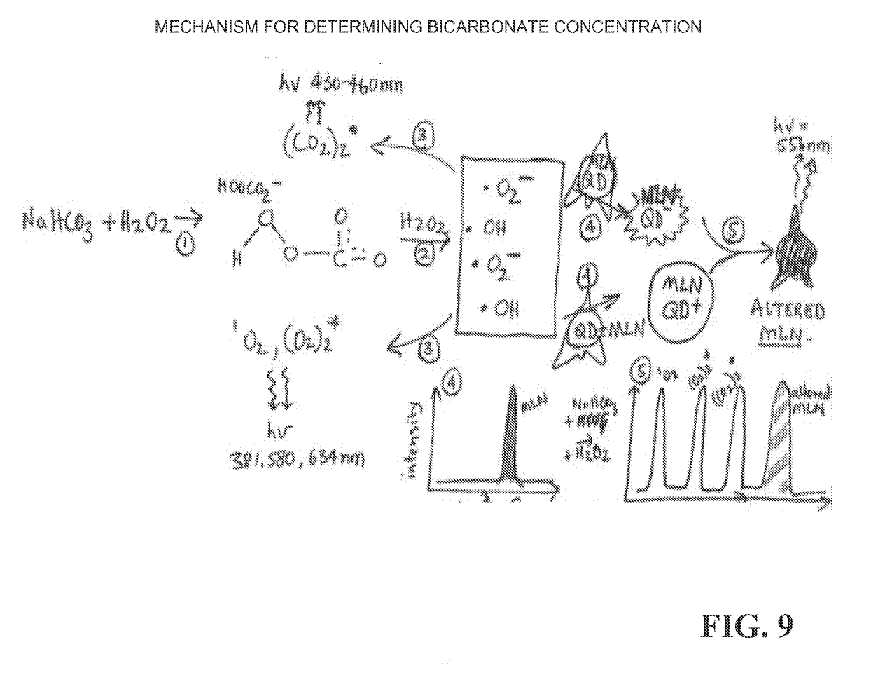

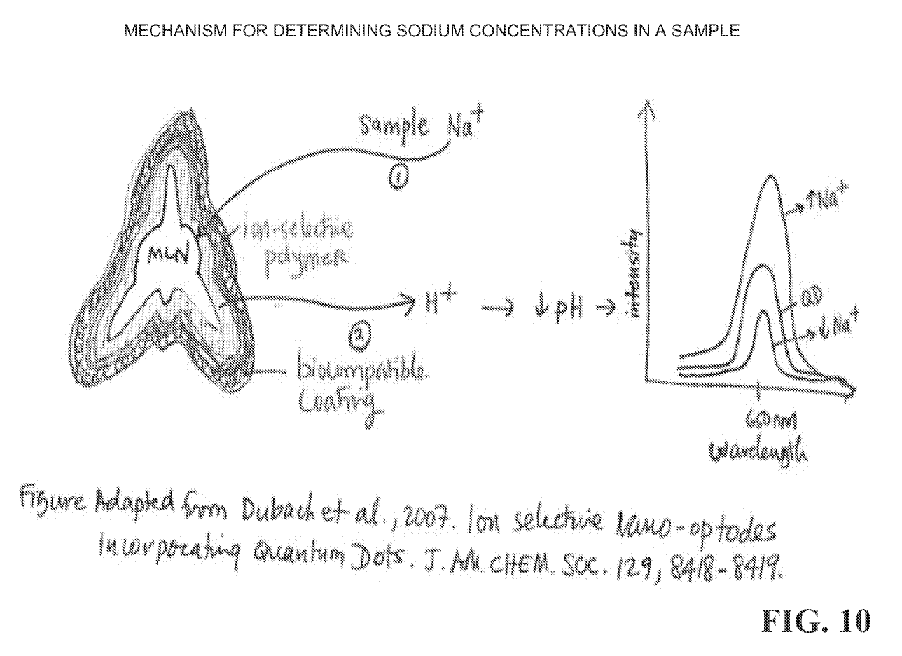

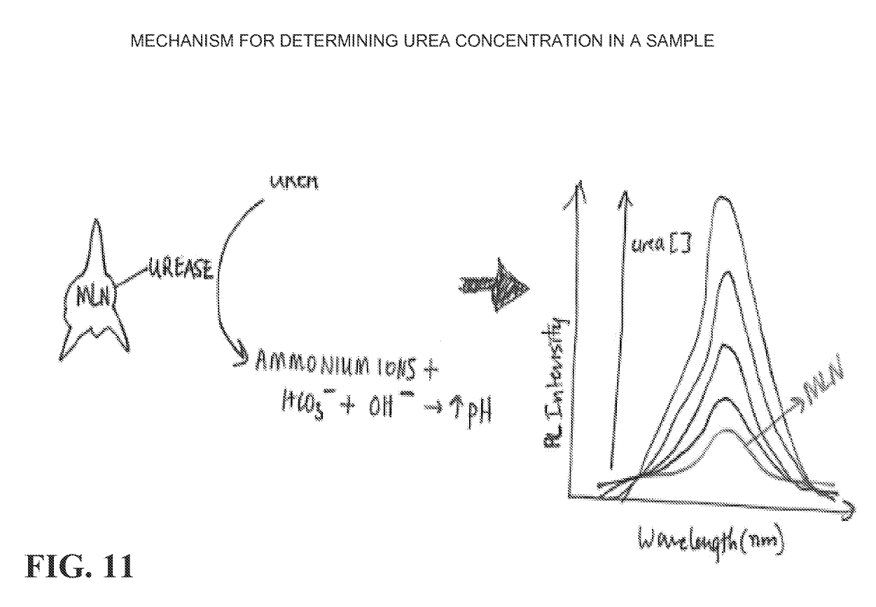

Referring now to FIG. 9, a non-limiting example of a mechanism for determining urea concentration in a sample is shown. A nanomaterial label, such as, MLN is paired to urease. In the presence of urea, the urease is cleaved into ammonium ions, hydroxyl ions, and HCO3, all of which correspond to an increase in pH levels. Upon the presence of Urea, the MLN emission spectrum will shift as compared to the emission spectrum of the MLN in the absence of urea. This shift will be detected by detection component and corresponds to a specific concentration of urea in the biological sample.

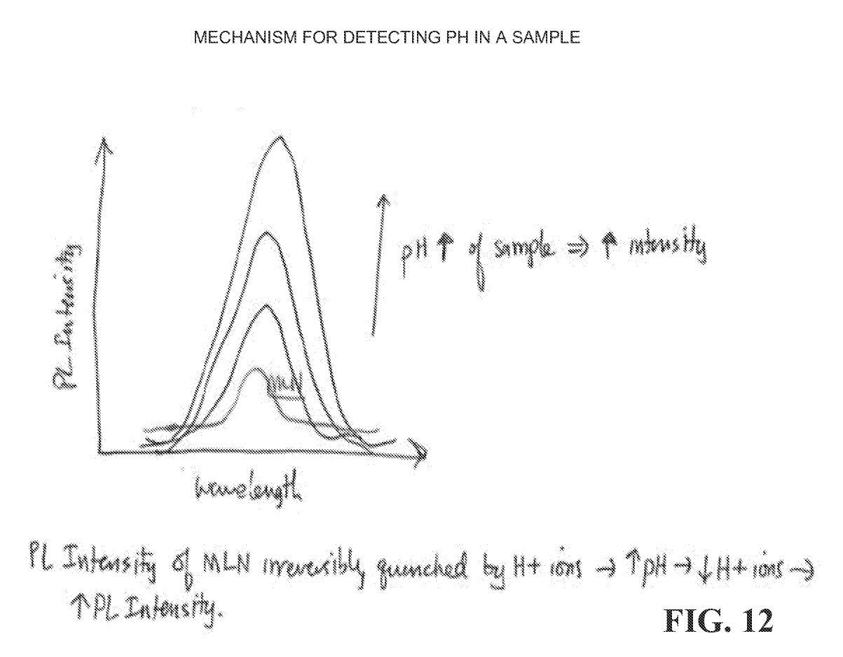

Referring now to FIG. 12 a non-limiting example of a mechanism for detecting pH in a sample is shown. In an aspect, an nanomaterial label in the presence of hydrogen ions can possess a quenched fluorescence. Thus an increase in pH corresponds to a decrease in hydrogen ions, which corresponds to an increase in photoluminescence intensity. Thus, the more pH in a sample, the greater the luminescence of the nanomaterial label such as the MLN as compared to the luminescence of the MLN in the absence of a biological sample and thus accordingly the absence of pH.

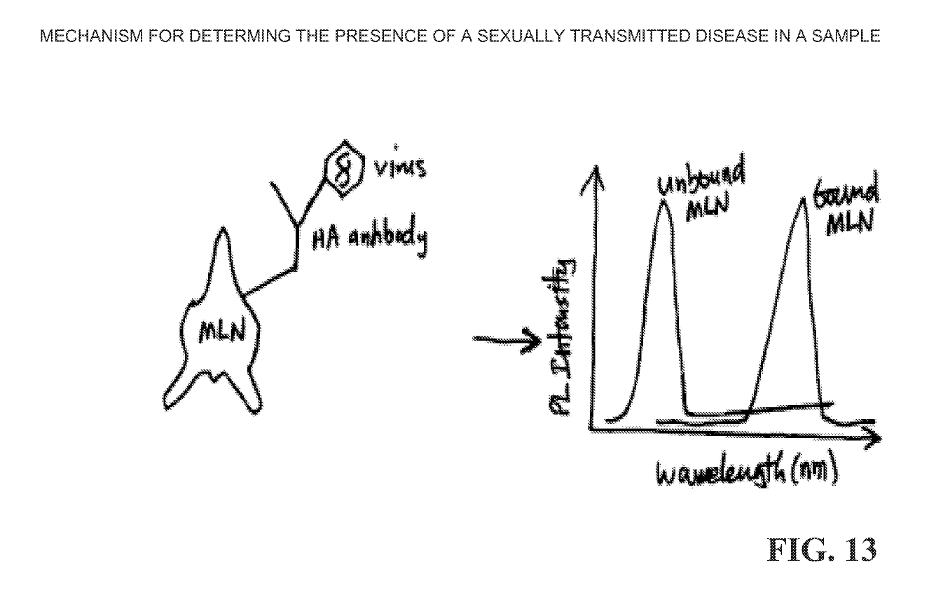

Referring now to FIG. 13, a non-limiting example of a mechanism for determining the presence of a sexually transmitted disease in a biological sample is shown. In an aspect, a nanomaterial label paired to an HA antibody can detect the presence of a virus. There is a change in the photoluminescence of the nanomaterial label, such as an MLN, in the presence of the virus than as compared to the MLN in the absence of the virus. The change in fluorescence (from an MLN in the absence of a virus as compared to art MLN in the presence of a virus) as depicted by a shift in wavelength's can be detected by detection component.

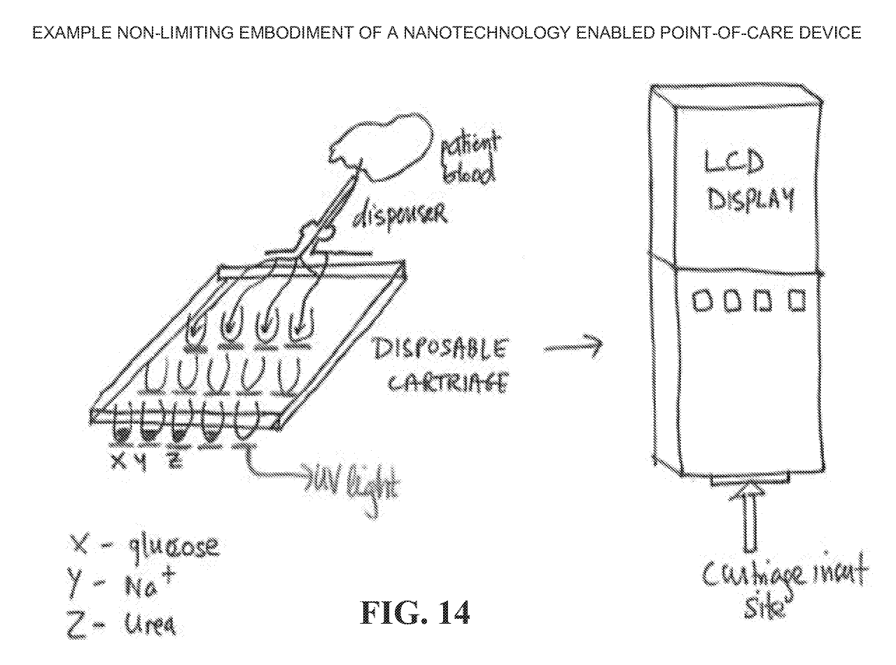

Referring now to FIG. 14, a non-limiting example of a non-limiting embodiment of a nanotechnology enabled point-of care device. In an aspect, a dispensing component dispenses blood from a patient sample into a disposable cartridge with multiple rows of wells. In each individual wells are different biological materials such as glucose in well “x”, sodium in well “y”, and urea in well “z.” An excitation source (e.g. via arm excitation component), such as UV light, can be applied to the wells from the bottom of the well or the top of the well whereby the UV light excites the nanomaterial labels paired to the respective biological materials in each individual well. The disposable cartridge can be input into a slot prior to dispersion of the patient biological sample whereby the device detects the fluorescence of the biological material paired to the nanomaterial label. Then, subsequent to dispersion of the patient biological sample into the individual wells, the cartridge component can be input into the device to, whereby the fluorescence of the nanomaterial label paired to the biological material paired to the biological target (e.g. urea, glucose, sodium), can be detected (e.g. via detection component), analyzed (e.g. via analysis component), generating a medical report (e.g. via generation component), and displayed on an LCD display screen in an embodiment (e.g. via, display component). Then after the reading the disposable cartridge can be disposed of.

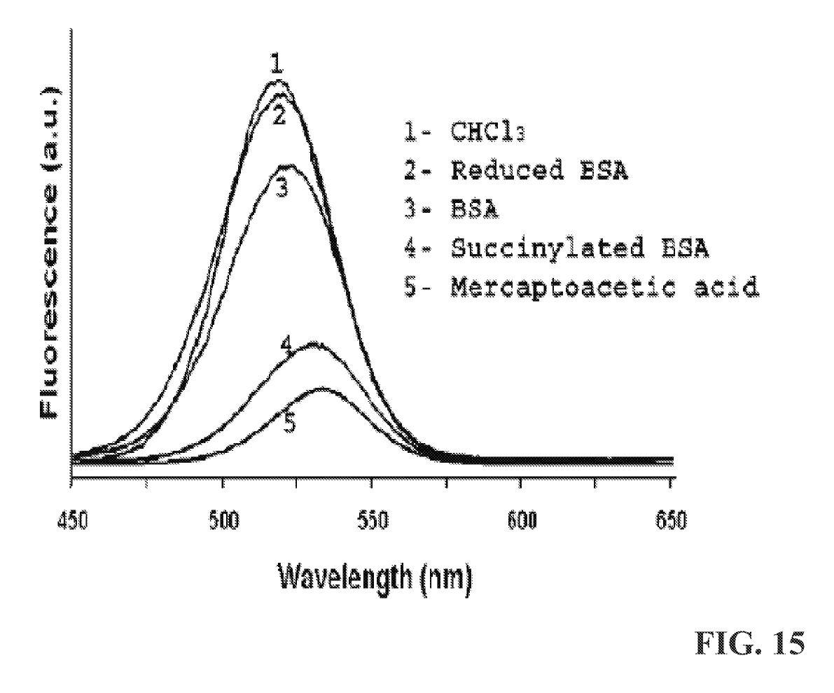

Referring now to FIG. 15, a non-limiting graph of the fluorescence spectra, of QD's with various ligands. Fluorescence spectra of water-soluble CdSe/ZnS quantum dots coated with chemically reduced BSA are shown in curve 1. Fluorescence spectra of water-soluble CdSe/ZnS quantum dots coated with standard BSA are shown in curve 2. Fluorescence spectra of water-soluble CdSe/ZnS quantum dots coated with succinylated BSA are shown in curve 4. Without BSA treatment, the fluorescence spectra of water-soluble dots (shown in curve 5) showed a 10 nm red shift in wavelength and were only ⅕ as bright as that of the original dots dissolved in chloroform (curve 1). With reduced BSA layer, both the fluorescence intensity and the spectral wavelength were restored to the original values.

Referring now to FIG. 16 is shown a mechanism of glucose sensing using a bienzyme hybrid system. The reaction results in the formation of benzoquinone and remove the necessity of enzyme immobilization and conjugation in the nanomaterial label.

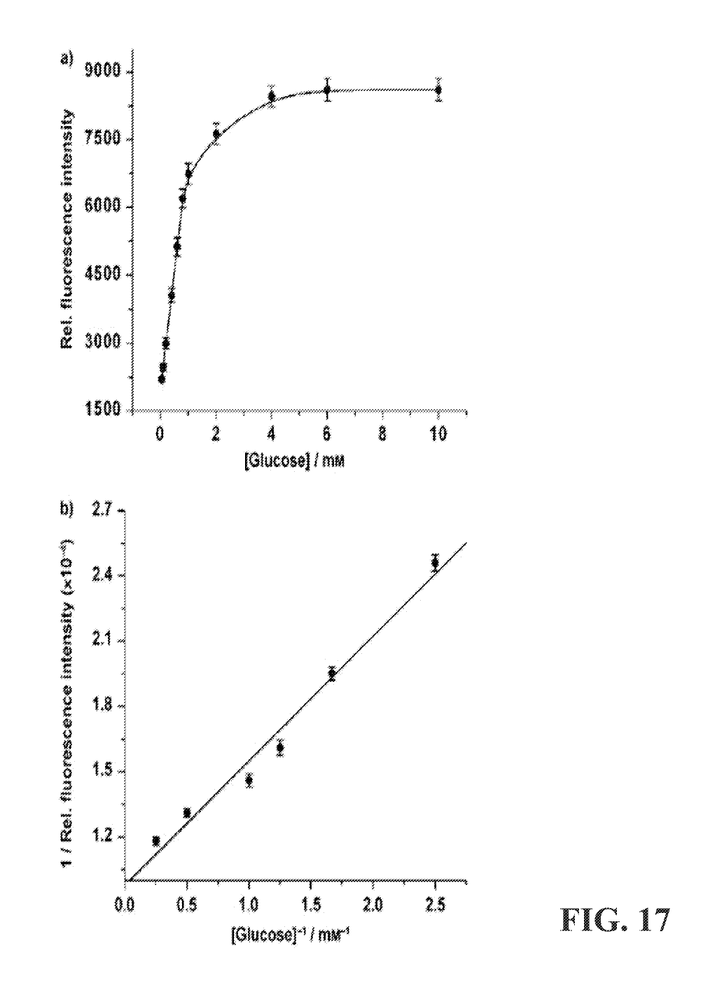

Referring now to FIG. 17 is shown a graphs indicating the calibration of CdTe-GOD system by Cao et, al., 2008. Shown in 17(a) is the relative fluorescence intensity changes after the addition of different concentrations of glucose to a sensing solution. Shown in 17(b) is the Lineweaver-Burke plots of the GOx enzyme-glucose reaction.

Referring now to FIG. 18 is shown a graph comparing the sensitivity with different capping agents on QD's. The PL spectra represented compares six sequential scans of GSH-capped QD's and MSA-capped QD's with the addition of 1 mM H2O2. The scan rate is 500 nm/min; scan delays are 3 minutes.

Referring now to FIG. 19 is shown a glucose biosensor based on nanocomposite film. In (A) is a scheme of sensing assembly; (a) is the Top 3 bilayers of PAH/GOD, (b) 3 bilayers of PAH/PSS, and (c) 12 are bilayers of PAH/CdTe QD's, (B) shows time-dependent fluorescence changes recorded at 630 nm upon the interaction of (PAH/CdTe)12 (PAH/PSS)3 (PAH/GOD)3 multilayer with variable concentrations of glucose: (a) 2, (b) 4, (c) 6, (d) 8, (e) 12, (f) 16, (g) 20, and (h) 40 mM. (C) Absolute quenching rate of the photoluminescence intensity taken from (B) within 5 minutes as a function of glucose concentration. F0 and Fm represent the photoluminescence intensity of (PAH/CdTe)12(PAH/GOD)3 multilayer at emission maximum in the absence (G0) and presence (Fm) of glucose.

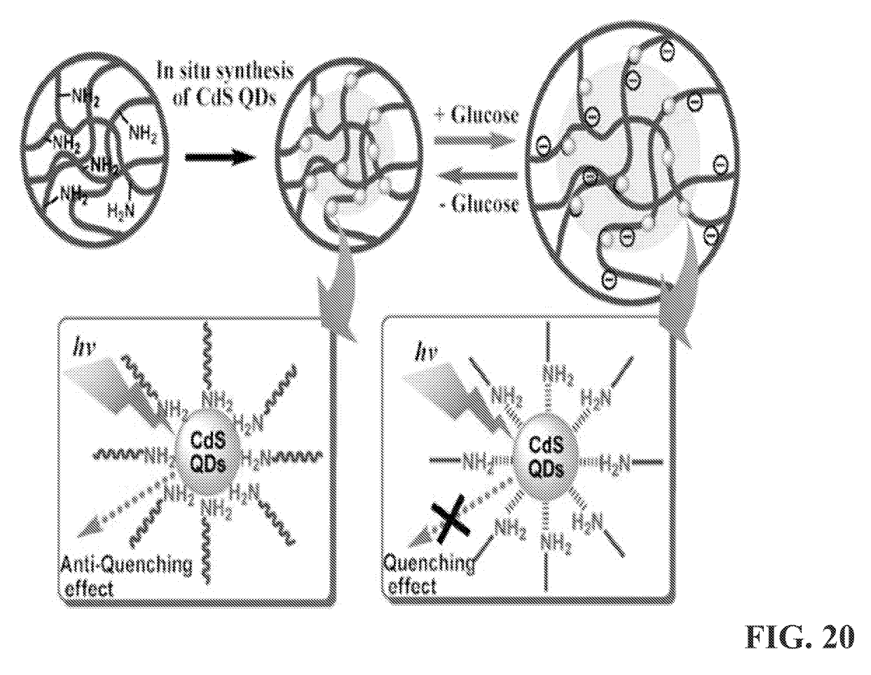

Referring now to FIG. 20 shown is glucose sensing by QD's immobilized in smart microgels. Reversible fluorescence quenching and anti-quenching of CdS QD's embedded in the interior of p (NIPAM-AAm-PBA) microgels in response to the change in glucose concentration, (a) Characteristic PL response of the p(NIPAM-AAm-PBA)-CdS hybrid microgels at 638 nm in the presence of D-glucose at pH=8.8.

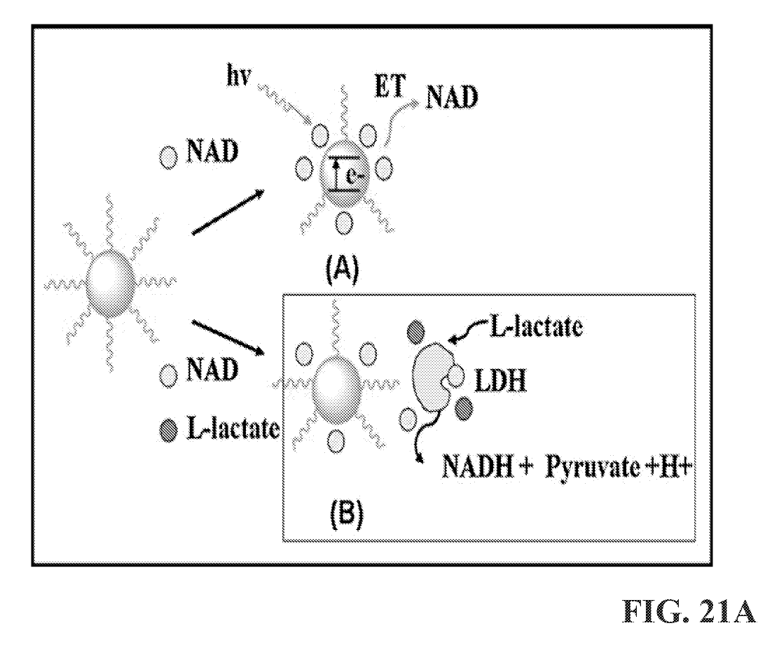

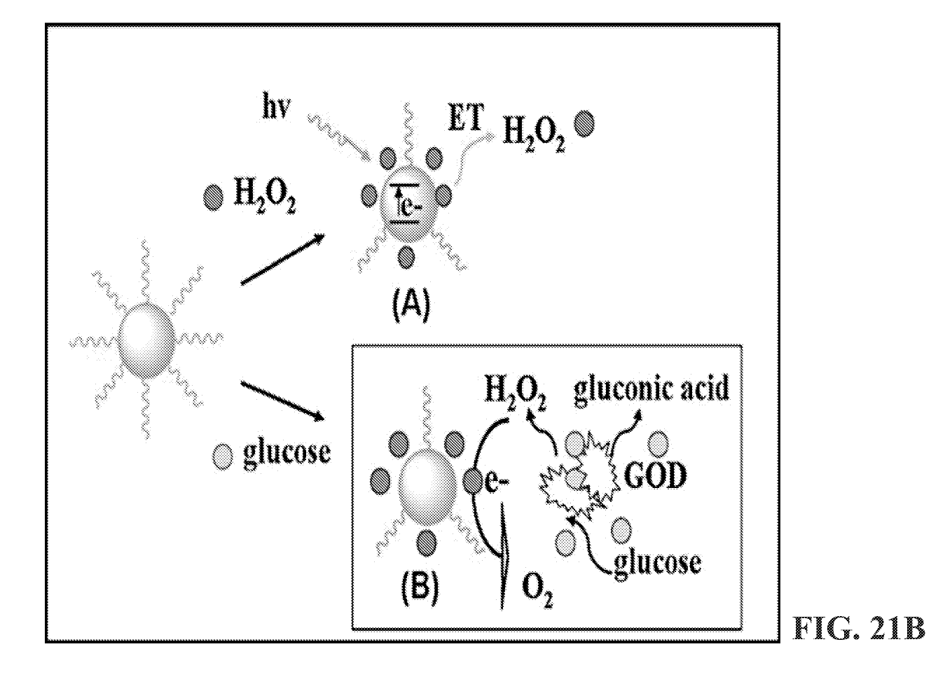

Referring now to FIG. 21 is shown a mechanism scheme of LDH and glucose sensing. The schematic principle for assay of NAD (A) and the activity of the LDH (B) based on the electron transfer of QD's and the biochemical reaction (left figure).

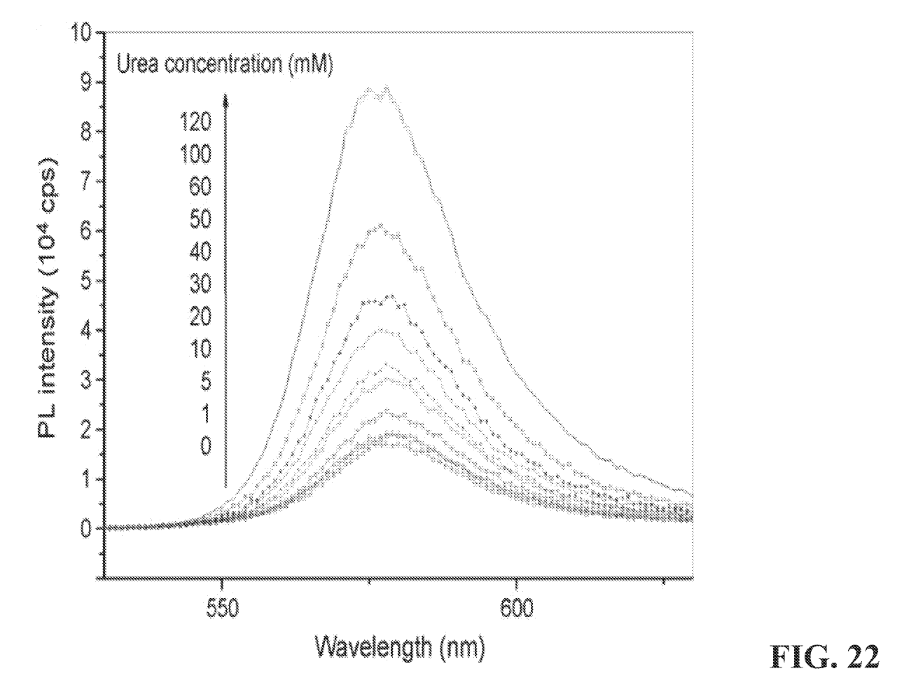

Referring now to FIG. 22 is shown a urea sensing using CdSe—ZnS core-shell quantum dots. The detection of urea, was based on fluorescence change as demonstrated by Huang C. P. et. al in 2006. When area is hydrolyzed to ammonium ions with the help of catalytic enzyme, urease, HCO3 ions and OH— are released and that causes the pH to increase. The results in the Figure were indicating the increase in fluorescence with a linear correlation to the increase in pH.

Referring now to FIG. 23 is shown the urea calibration curves reported by Duong and Rhee (2008). The linear plot for urea sensing using the QD-entrapped sol-gel membrane (left) is shown. The urea sensing using the urease immobilized sol-gel membrane (middle) is shown. The sensing of urea using the double layer of QD-trapped and urease immobilized sol-gel membrane (right) is shown.

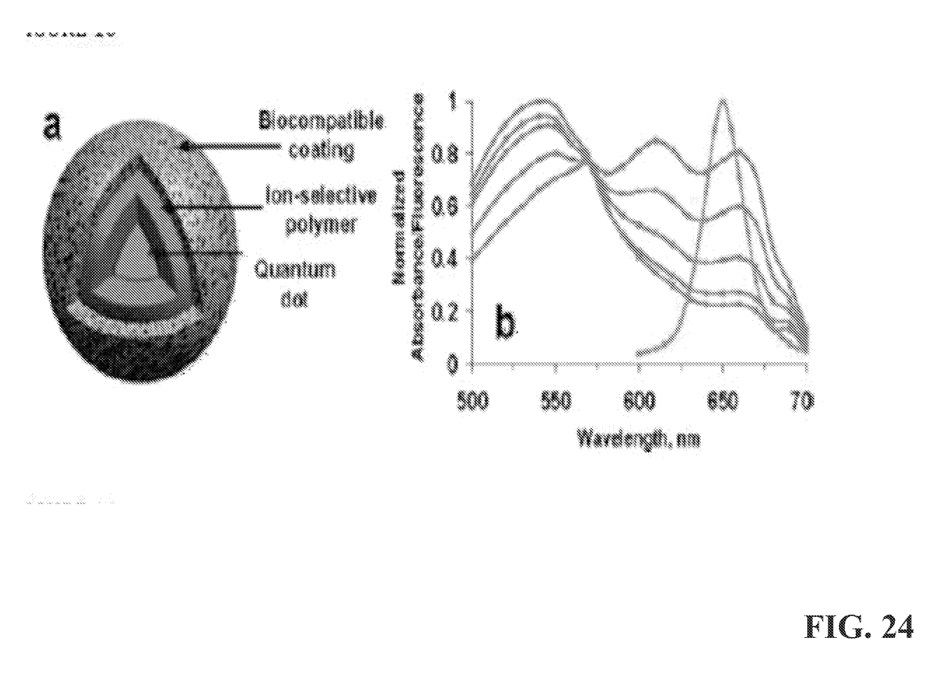

Referring now to FIG. 24, shown is sodium sensing using QD's. In (a) the assembly of the sodium nano-optode is shown. In (b) the absorption of chromoionophore (gray lines) and the emission spectra of the QD (red line) are shown.

Referring now to FIG. 25 is shown a response curves for sodium biosensing. In (a) shown is the experimental response of ion-selective QD's to sodium concentrations. In (b) shown is the calibration curve of ratiometric ISQD.

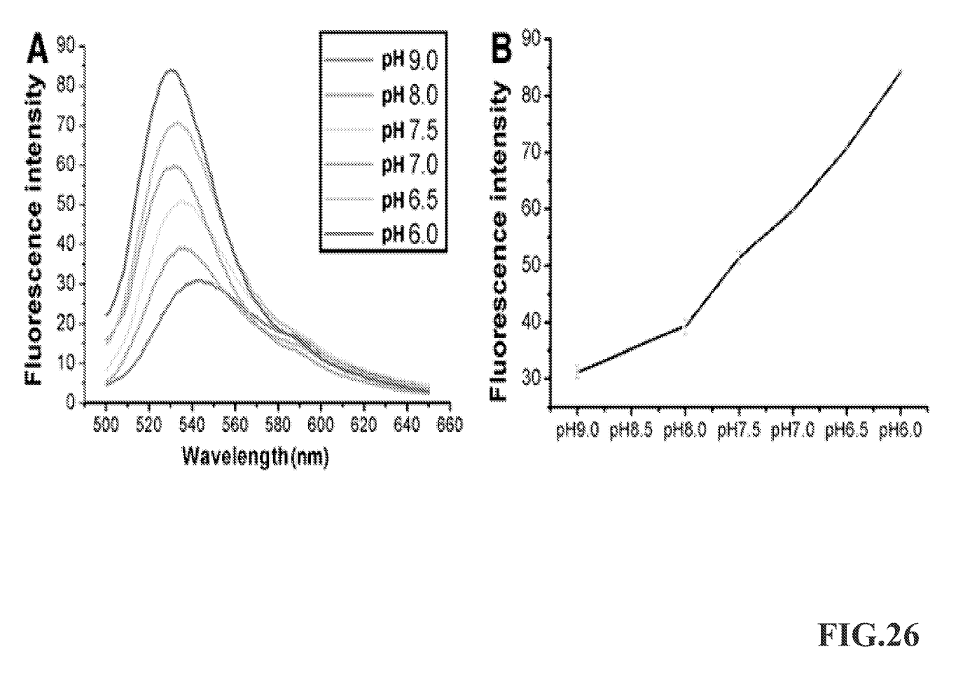

Referring now to FIG. 26 shown is the effect of pH on QD fluorescence in view to study enzyme kinetics. The emission spectrum of QD's to the variation of pH. A to H are 8.0, 7.5, 7.0, 6.5, 6.0, 5.5, 5.0, 4.5, respectively. Inset shows the variation of the FL intensity of QD's at 562 nm at various pH.

Referring now to FIG. 27 is shown the effect of pH on QD fluorescence in view of virus detection. At (A), shown is the variation of fluorescence with change in pH. At (B) shown is the Peak fluorescence intensity of each curve plotted against pH.

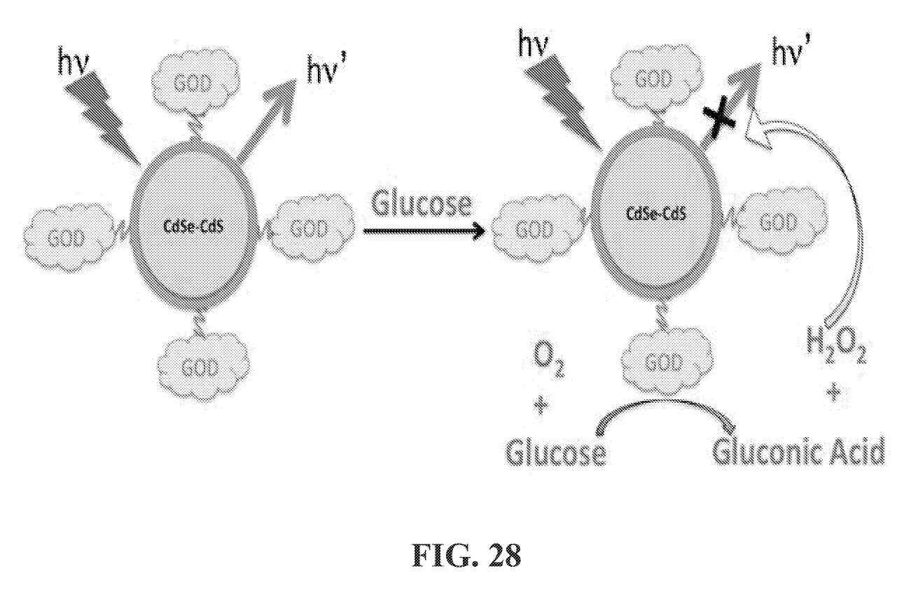

Referring now to FIG. 28 is shown a mechanism for glucose sensing. The H2O2 released during glucose oxidase enzyme acts as an electron acceptor, thus quenching the fluorescence of QD's.

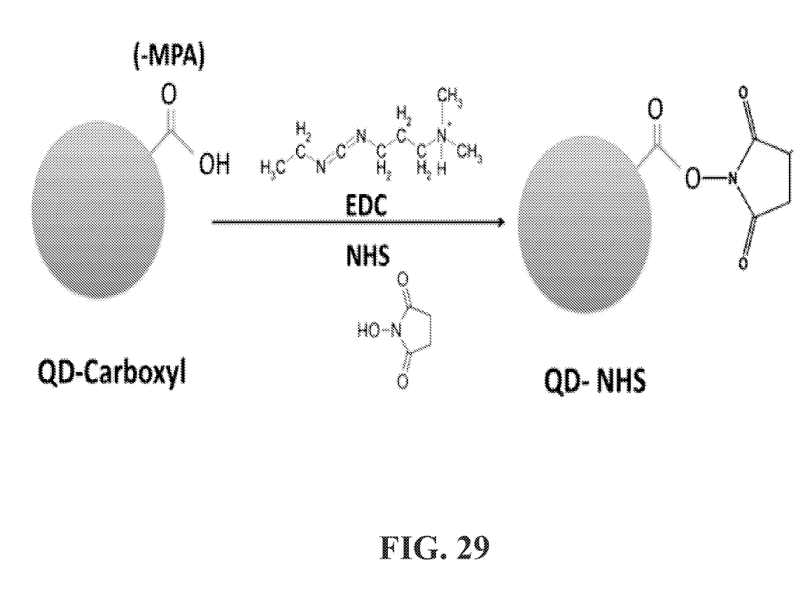

Referring now to FIG. 29 is shown a mechanism of conjugation of GOD with QD. The conjugation of Glucose Oxidase (GOD) to QD's using EDC and NHS as the cross-linkers, giving an amide linkage between the QD and GOD.

Referring now to FIG. 30 is shown equations for the mechanism of activity estimation. The enzyme activity was obtained for 0, 2, and 4 weeks, thus giving the stability of GOD conjugated on the QD surface. Quantification of this stability is represented by a parameter Enzyme Stability Coefficient (ESC), defined as the percentage of activity retained by the QD-ligand-GOD system after keeping samples at room temperature for four weeks.

Referring now to FIG. 31 are shown the properties of CdSe, CdS, ZnS semiconductors used in the glucose study.

Referring now to FIG. 32 is shown TEM micrographs of QD's. TEM micrographs for CdSe—ZnS (MAA (A), MPA (B), GSH (C)), CdSe—CdS (MAA (D), MPA (E), GSH (F)) and CdS—ZnS (MAA (G), MPA (H), GSH (I)).

Referring now to FIG. 33 is shown a HRTEM micrographs of QD's. HRTEM micrographs and ED patterns for CdSe—ZnS (A and D), CdSe—CdS (B and E) and CdS—ZnS (C and F). Scale bar=5 nm for all the HRTEM micrographs and Scale bar=10000000 nm for all the ED patterns.

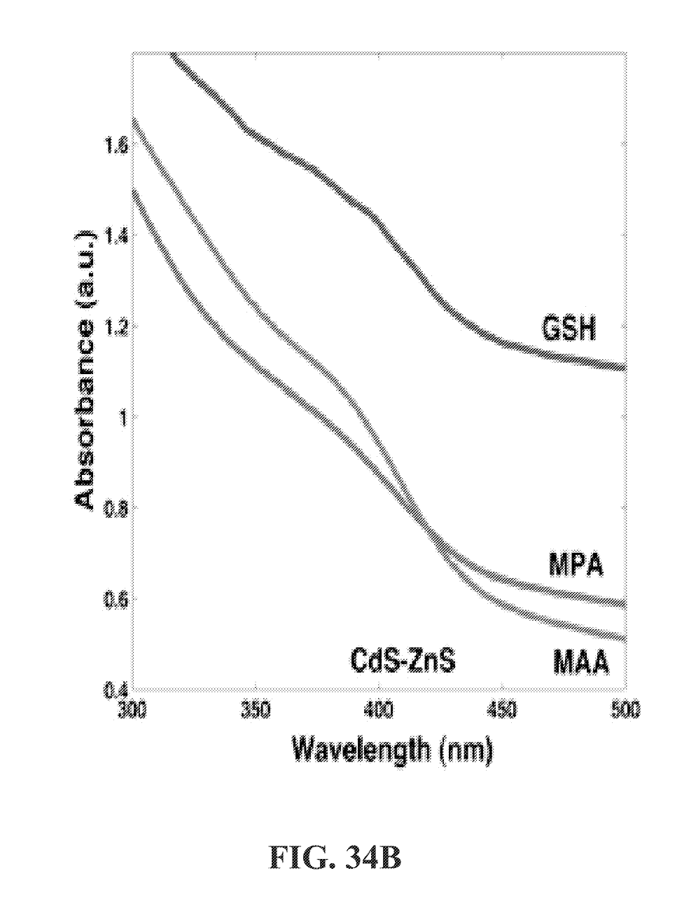

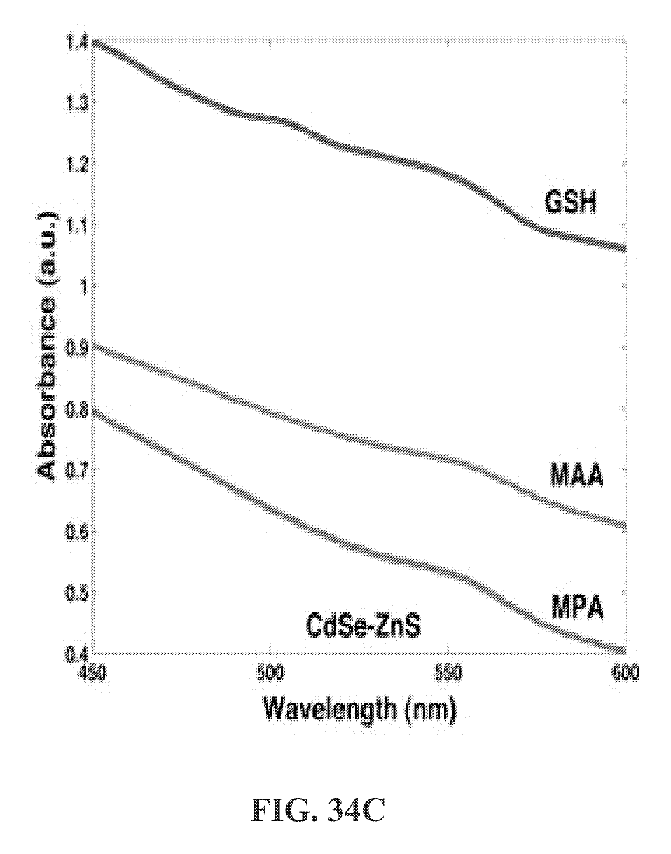

Referring now to FIG. 34 is shown the size distribution and analysis of QD-ligand systems. The absorbance spectra for CdSe—ZnS, CdSe—CdS and CdS—ZnS for the three ligands MAA, MPA and GSH. Comparison of mean particle size (nm) for the nine systems from TEM and Absorbance.

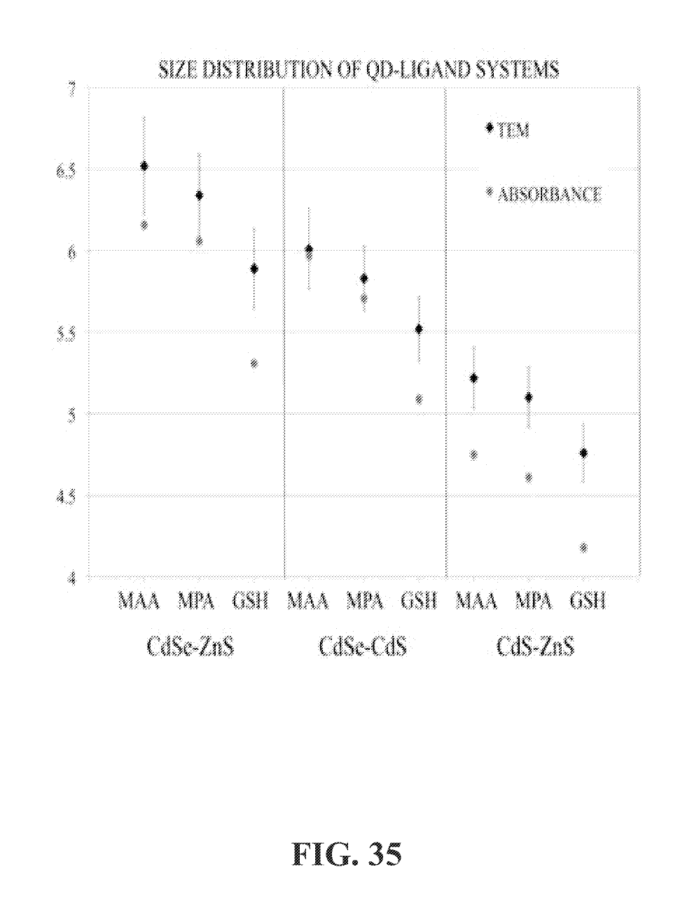

Referring now to FIG. 35 shown is Fluorescence of spectra and its response to H2O2. Fluorescence spectra for CdSe—ZnS (MAA (A), MPA (B), GSH (C)), CdSe—CdS (MAA (D), MPA (E), GSH (F)) and CdS—ZnS (MAA (G), MPA (H), GSH (I)). The spectra are recorded for varying concentrations of H2O2 (as shown in side bar) at an excitation wavelength of 390 mm. The S value shown under each panel is the slope of Relative Intensity (I/I0) versus H2O2 concentration. The Sensitivity Index (SI) is defined here as the modulus of the slope S.

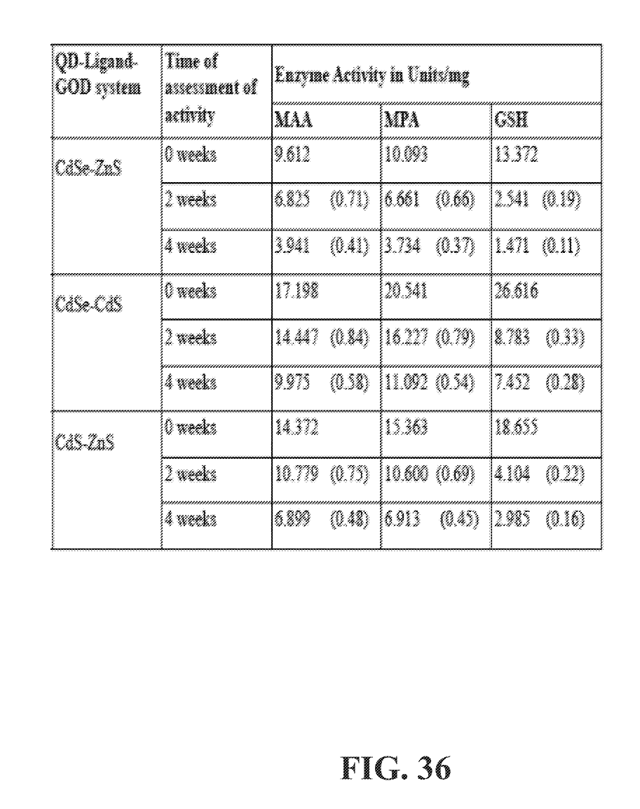

Referring now to FIG. 36 is shown a table depicting enzyme activity for the nine QD-ligand systems estimated after 0, 2 and 4 weeks, with samples kept at room temperature.

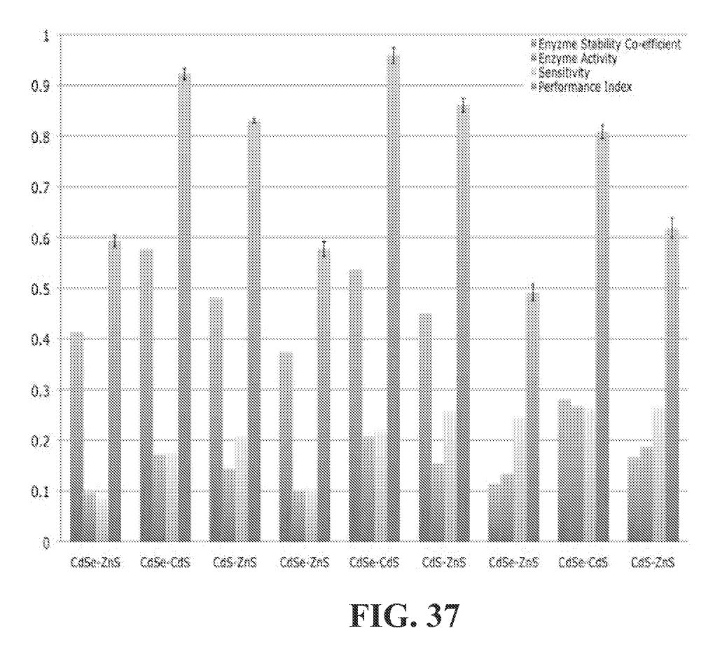

Referring now to FIG. 37 is shown the evaluation of QD-ligand systems for performance as biosensors. The scaled values for the three parameters, (SI, EA, and ESC) are shown for the nine QD-ligand systems. The resultant Performance Index (PI) values are shown at the top of each system.

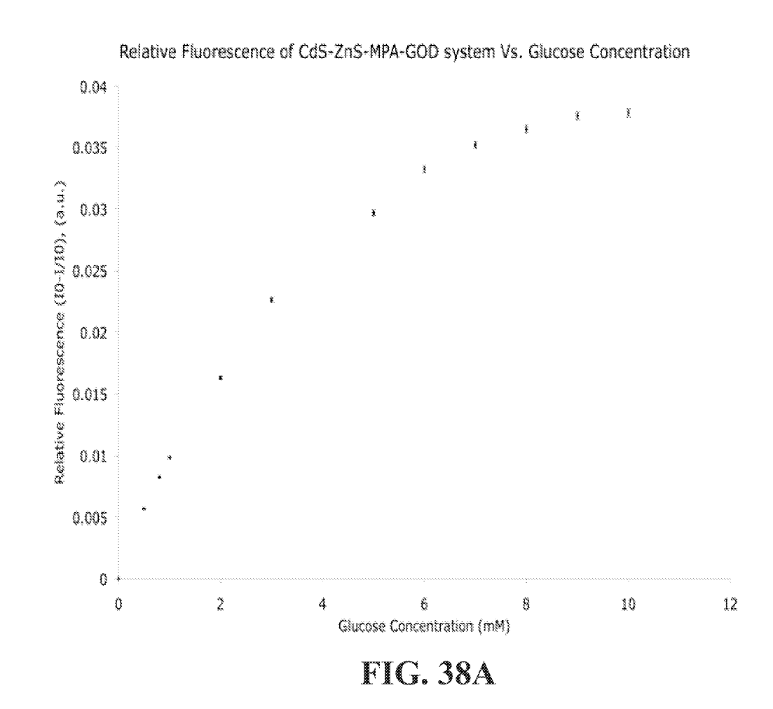

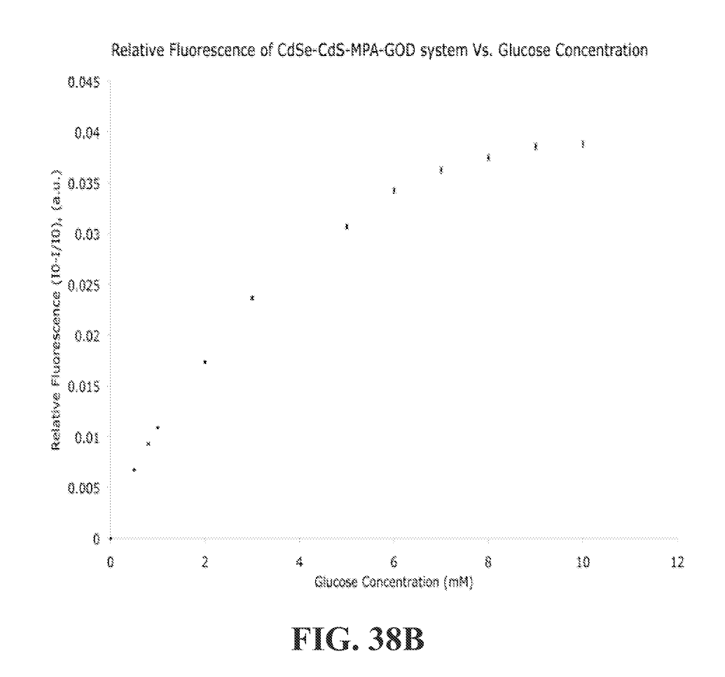

Referring now to FIG. 38 is shown the relative fluorescence of the QD-ligand systems to various concentrations of glucose. This fluorescence is taken with the QD-ligand system centrifuged and washed to remove excess GOD.

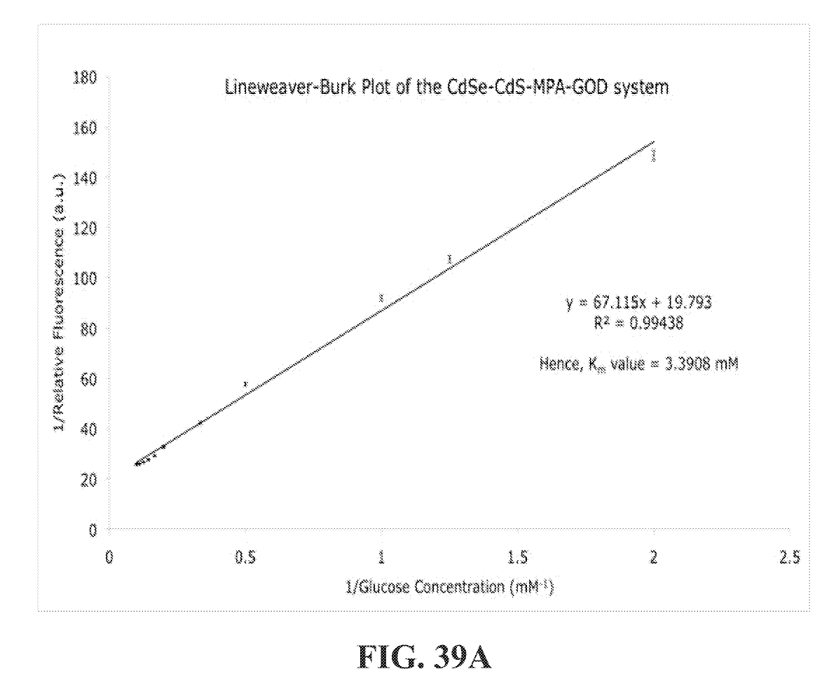

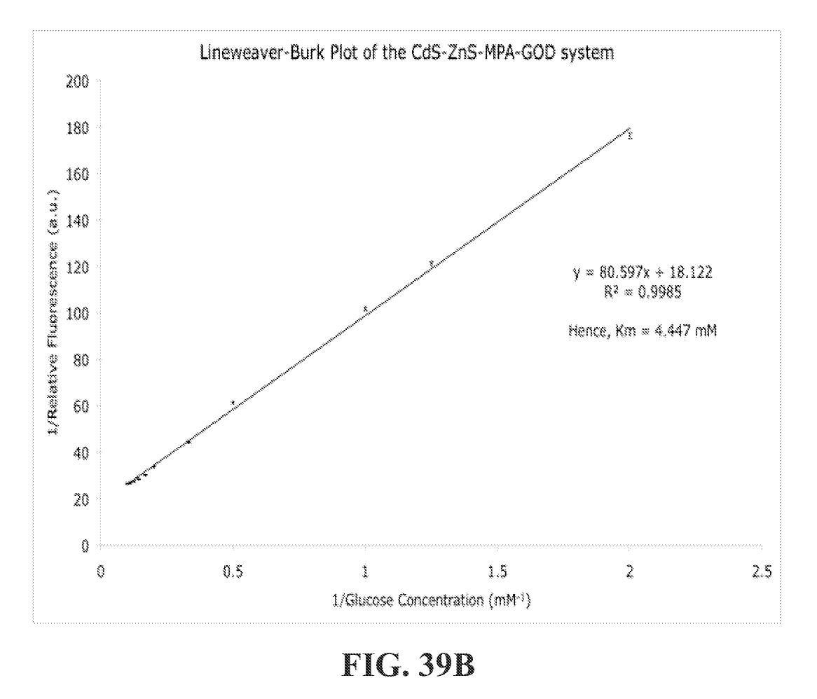

Referring now to FIG. 39 is shown modified Lineweaver-Burke plots of the QD-ligand systems. These are plots of the fluorescence taken with the QD-ligand system centrifuged and washed to remove excess GOD.

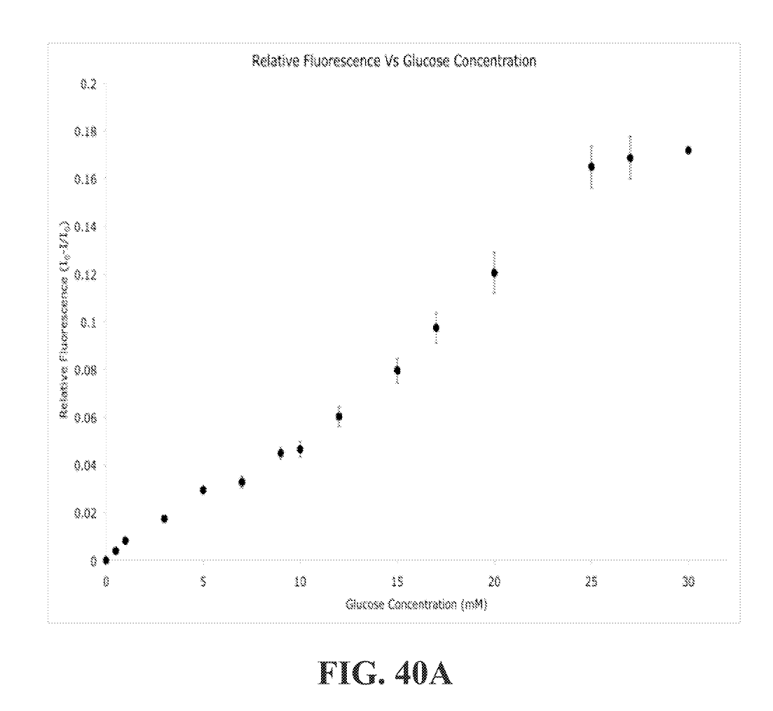

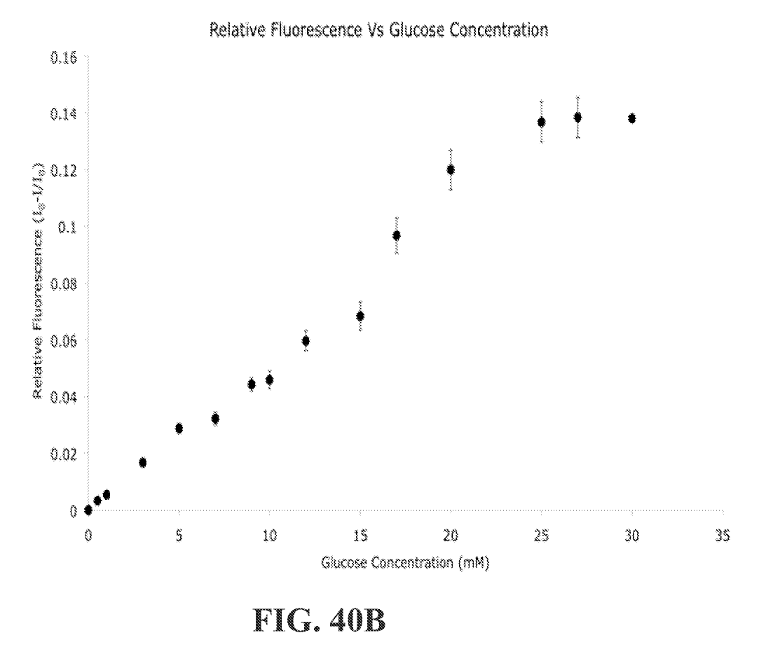

Referring now to FIG. 40, is shown relative fluorescence of the QD-ligand systems (uncentrifuged) to various concentrations of glucose. This fluorescence is taken with the QD-ligand system without centrifugation. At the left is CdSe—CdS-MPA-GOD and at the right, is the CdS—ZnS-MPA-GOD.

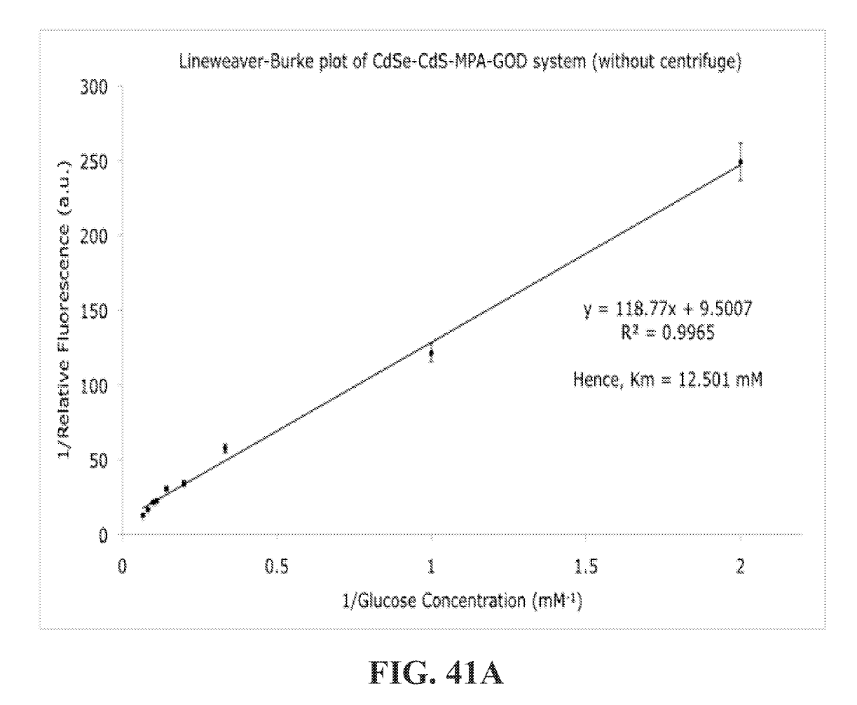

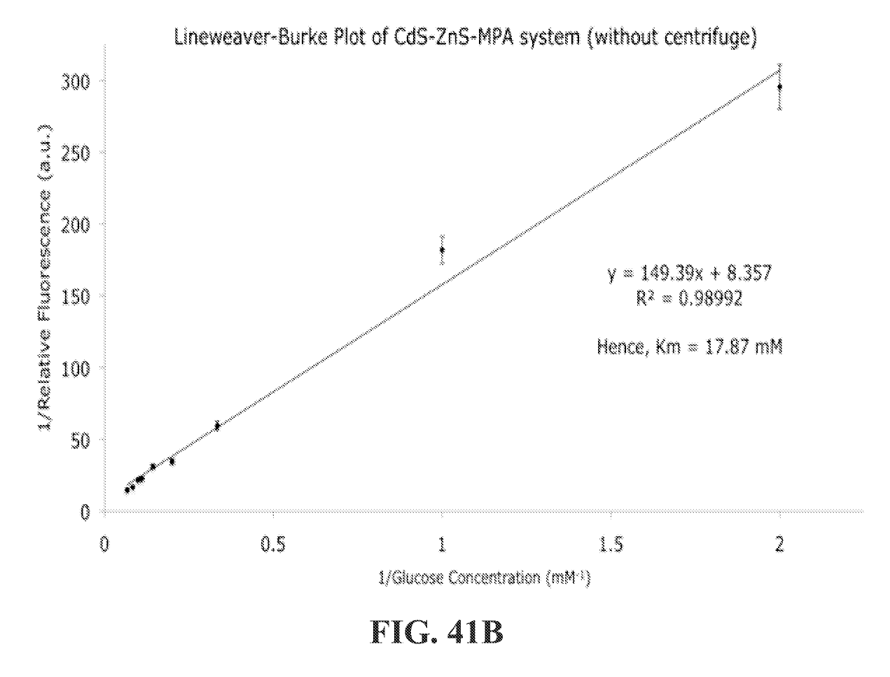

Referring now to FIG. 41 is shown modified Lineweaver-Burke pits of the QD-ligand systems (uncentrifuged). These are plots of the fluorescence taken with the QD-ligand system uncentrifuged.

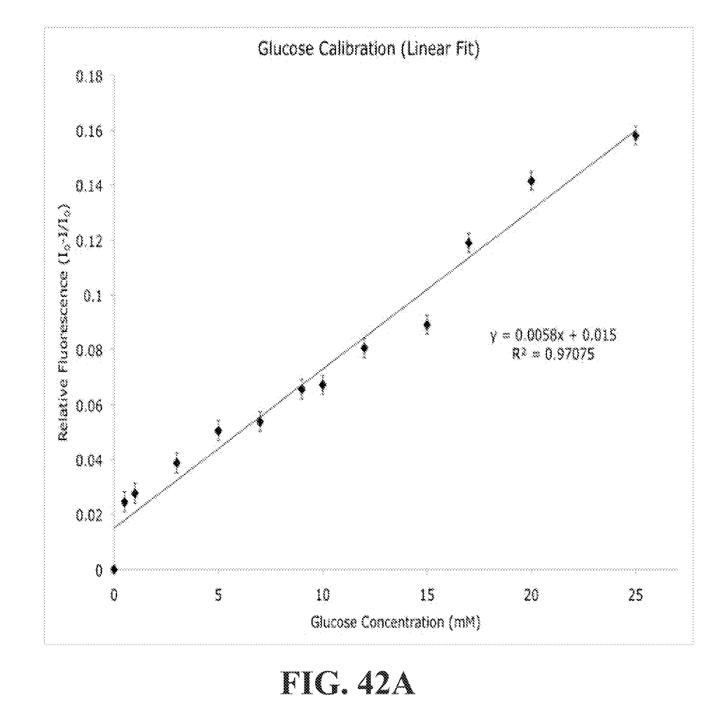

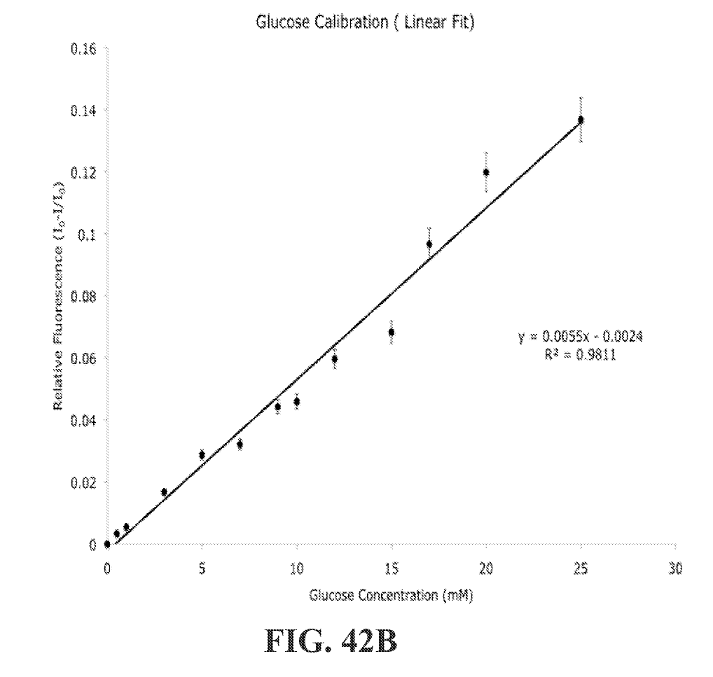

Referring now to FIG. 42 is shown linear fit models for glucose estimation. At the left is CdSe—CdS-MPA-GOD linear fit model and at the right is CdS—ZnS-MPA-GOD linear fit model.

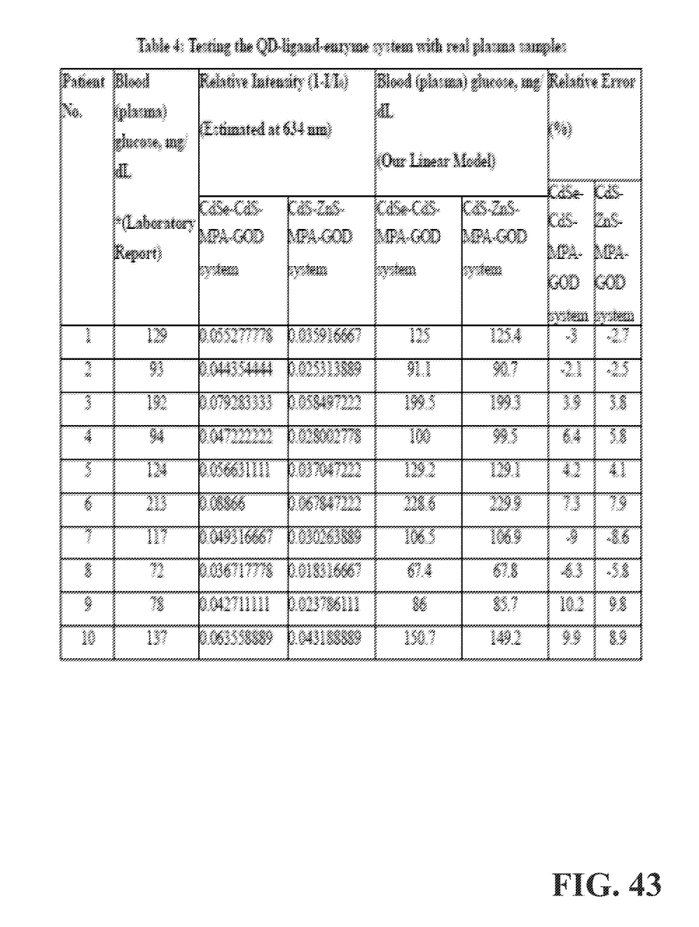

Referring now to FIG. 43 is shown a table of the testing of the QD-ligand-enzyme system with real plasma samples.

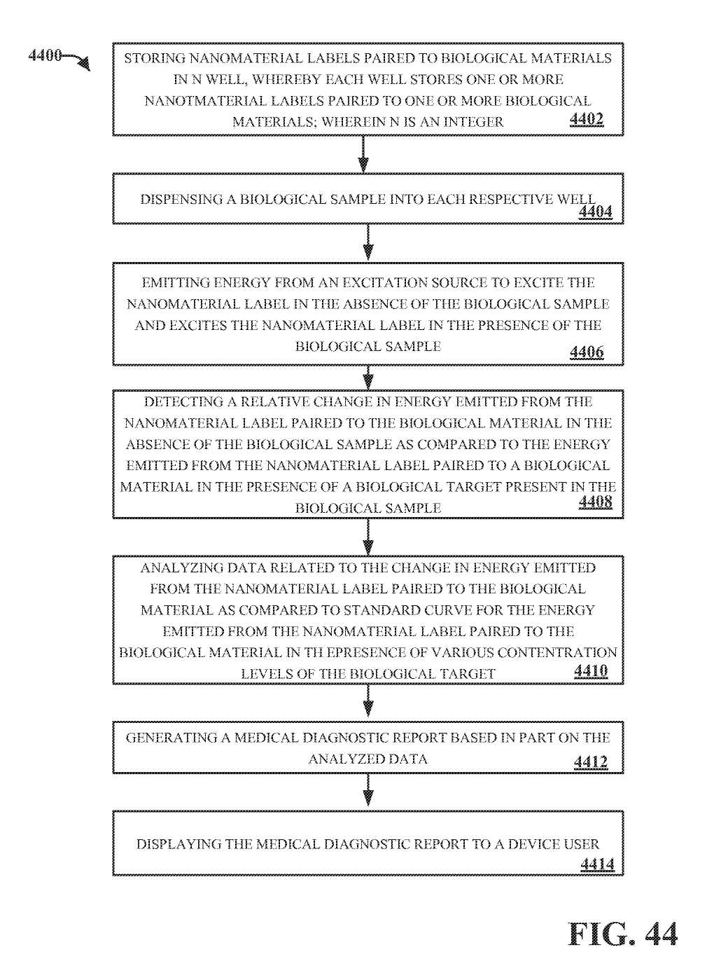

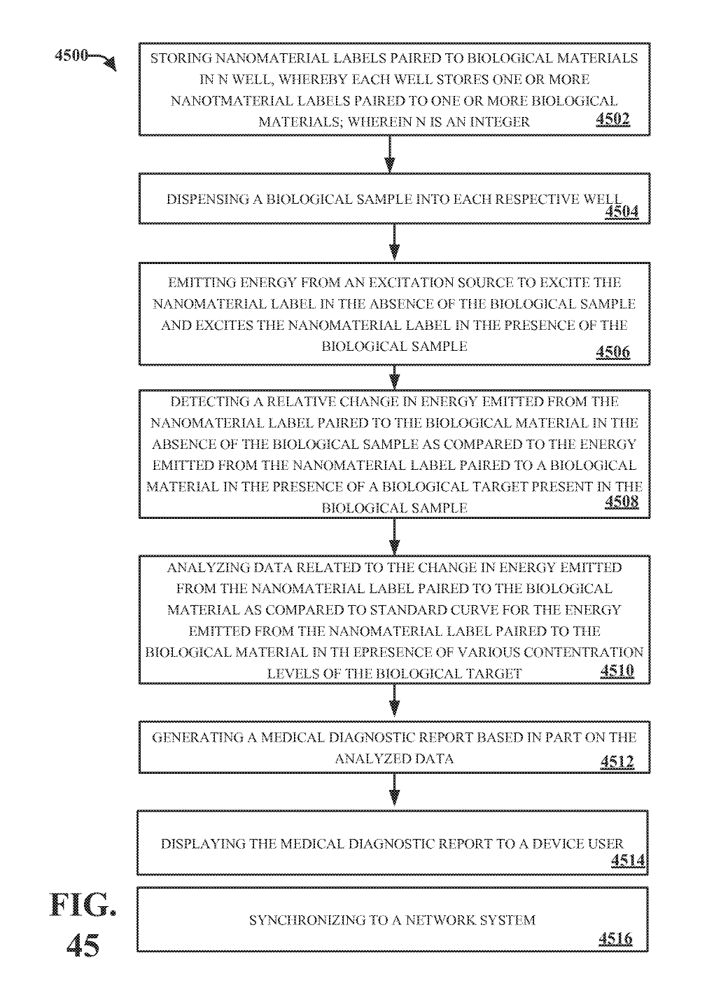

FIGS. 44-45 illustrates a methodology or flow diagram in accordance with certain aspects of this disclosure. While, for purposes of simplicity of explanation, the methodologies are shown and described as a series of acts, the disclosed subject matter is not limited by the order of acts, as some acts may occur in different orders and/or concurrently with other acts from that shown and described herein. For example, those skilled in the art will understand and appreciate that a methodology can alternatively be represented as a series of interrelated states or events, such as in a state diagram. Moreover, not all illustrated acts may be required to implement a methodology in accordance with the disclosed subject matter. Additionally, it is to be appreciated that the methodologies disclosed in this disclosure are capable of being stored on an article of manufacture to facilitate transporting and transferring such methodologies to computers or other computing devices.

Referring now to FIG. 44, presented is a flow diagram of an example application of a point of care device disclosed in this description in accordance with an embodiment. In an aspect, exemplary methodology 4400 of a point of care device is stored in a memory and utilizes a processor to execute computer executable instructions to perform functions. At 4402, point of care device stores (e.g. using cartridge component 110) nanomaterial labels paired to biological materials in N wells, whereby each well stores one or more nanomaterial labels paired to one or more biological materials; wherein N is an integer. At 4404, point of care device dispenses (e.g. using dispensing component 120) a biological sample into each respective well.

At 4406, point of care device emits (e.g. using excitation component 130) energy from an excitation source to excite the nanomaterial label in the absence of the biological sample and excites the nanomaterial label in the presence of the biological sample. At 4408, point of care device detects (e.g. using detection component 140) a relative change in energy emitted from the nanomaterial label paired to the biological material in the absence of the biological sample as compared to the energy emitted from the nanomaterial label paired to a biological material in the presence of a biological target present in the biological sample.

At 4410, point of care device analyzes data (e.g. using analysis component 150) related to the change in energy emitted from the nanomaterial label paired to the biological material as compared to a standard curve for the energy emitted from the nanomaterial label paired to the biological material in the presence of various concentration levels of the biological target. At 4412, point of care device generates (e.g. using a generation component 160) a medical diagnostic report based in part on the analyzed data. At 4414, point of care device displays (e.g. using display component 170) the medical diagnostic report to a device user.

Referring now to FIG. 45, presented is a flow diagram of an example application of a point of care device disclosed in this description in accordance with an embodiment. In an aspect, exemplary methodology 4500 of a point of care device is stored in a memory and utilizes a processor to execute computer executable instructions to perform functions. At 4502, point of care device stores (e.g. using cartridge component 110) nanomaterial labels paired to biological materials in N wells, whereby each well stores one or more nanomaterial labels paired to one or more biological materials; wherein N is an integer. At 4504, point of care device dispenses (e.g. using dispensing component 120) a biological sample into each respective well.

At 4506, point of care device emits (e.g. using excitation component 130) energy from an excitation source to excite the nanomaterial label in the absence of the biological sample and excites the nanomaterial label in the presence of the biological sample. At 4508, point of care device detects (e.g. using detection component 140) a relative change in energy emitted from the nanomaterial label paired to the biological material in the absence of the biological sample as compared to the energy emitted from the nanomaterial label paired to a biological material in the presence of a biological target present in the biological sample.

At 4510, point of care device analyzes data (e.g. using analysis component 150) related to the change in energy emitted from the nanomaterial label paired to the biological material as compared to a standard curve for the energy emitted from the nanomaterial label paired to the biological material in the presence of various concentration levels of the biological target. At 4512, point of care device generates (e.g. using a generation component 160) a medical diagnostic report based in part on the analyzed data. At 4514, point of care device displays (e.g. using display component 170) the medical diagnostic report to a device user. At 4516, point of care device synchronizes (e.g. using synchronization component 310) the device to a network system or another device.

### Example Operating Environments

The systems and processes described below can be embodied within a device via hardware, such as a single integrated circuit (IC) chip, multiple ICs, an application specific integrated circuit (ASIC), or the like. Further, the order in which some or all of the process blocks appear in each process should not be deemed limiting. Rather, it should be understood that some of the process blocks can be executed in a variety of orders, not all of which may be explicitly illustrated in this disclosure.

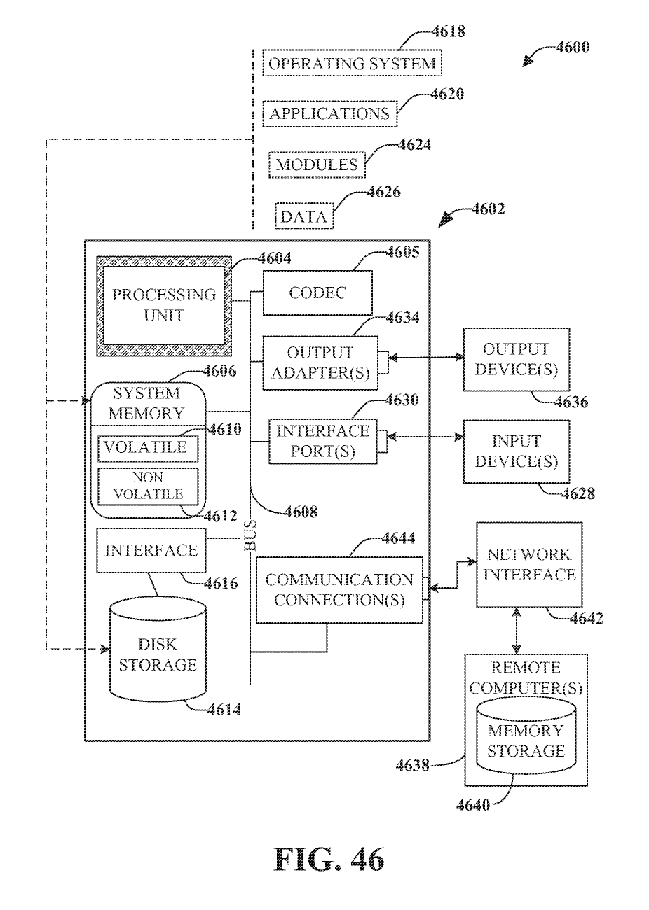

With reference to FIG. 46, a suitable environment 4600 for implementing various aspects of the claimed subject matter includes a computer 4602. The computer 4602 includes a processing unit 4604, a system memory 4606, a codec 4605, and a system bus 4608. The system bus 4608 couples system components including, but not limited to, the system memory 4606 to the processing unit 4604. The processing unit 4604 can be any of various available processors. Dual microprocessors and other multiprocessor architectures also can be employed as the processing unit 4604.

The system bus 4608 can be any of several types of bus structure(s) including the memory bus or memory controller, a peripheral bus or external bus, and/or a local bus using any variety of available bus architectures including, but not limited to, Industrial Standard Architecture (ISA), Micro-Channel Architecture (MSA), Extended ISA (EISA), Intelligent Drive Electronics (IDE), VESA Local Bus (VLB), Peripheral Component Interconnect (PCI), Card Bus, Universal Serial Bus (USB), Advanced Graphics Port (AGP), Personal Computer Memory Card International Association bus (PCMCIA), Firewire (IEEE 1394), and Small Computer Systems Interface (SCSI).

The system memory 4606 includes volatile memory 4610 and non-volatile memory 4612. The basic input/output system (BIOS), containing the basic routines to transfer information between elements within the computer 4602, such as during start-up, is stored in non-volatile memory 4612. In addition, according to present innovations, codec 4605 may include at least one of an encoder or decoder, wherein the at least one of an encoder or decoder may consist of hardware, a combination of hardware and software, or software. Although, codec 4605 is depicted as a separate component, codec 4605 may be contained within non-volatile memory 4612. By way of illustration, and not limitation, non-volatile memory 4612 can include read only memory (ROM), programmable ROM (PROM), electrically programmable ROM (EPROM), electrically erasable programmable ROM (EEPROM), or flash memory. Volatile memory 4610 includes random access memory (RAM), which acts as external cache memory. According to present aspects, the volatile memory may store the write operation retry logic (not shown in FIG. 46) and the like. By way of illustration and not limitation, RAM is available in many forms such as static RAM (SRAM), dynamic RAM (DRAM), synchronous DRAM (SDRAM), double data rate SDRAM (DDR SDRAM), and enhanced SDRAM (ESDRAM.

Computer 4602 may also include removable/non-removable, volatile/non-volatile computer storage medium. FIG. 46 illustrates, for example, disk storage 4614. Disk storage 4614 includes, but is not limited to, devices like a magnetic disk drive, solid state disk (SSD) floppy disk drive, tape drive, Jaz drive, Zip drive, LS-70 drive, flash memory card, or memory stick. In addition, disk storage 4614 can include storage medium separately or in combination with other storage medium including, but not limited to, an optical disk drive such as a compact disk ROM device (CD-ROM), CD recordable drive (CD-R Drive), CD rewritable drive (CD-RW Drive) or a digital versatile disk ROM drive (DVD-ROM). To facilitate connection of the disk storage devices 4614 to the system bus 4608, a removable or non-removable interface is typically used, such as interface 4616.

It is to be appreciated that FIG. 46 describes software that acts as an intermediary between users and the basic computer resources described in the suitable operating environment 4600. Such software includes an operating system 4618. Operating system 4618, which can be stored on disk storage 4614, acts to control and allocate resources of the computer system 4602. Applications 4620 take advantage of the management of resources by the operating system through program modules 4624, and program data 4626, such as the boot/shutdown transaction table and the like, stored either in system memory 4606 or on disk storage 4614. It is to be appreciated that the claimed subject matter can be implemented with various operating systems or combinations of operating systems.

A user enters commands or information into the computer 4602 through input device(s) 4628. Input devices 4628 include, but are not limited to, a pointing device such as a mouse, trackball, stylus, touch pad, keyboard, microphone, joystick, game pad, satellite dish, scanner, TV tuner card, digital camera, digital video camera, web camera, and the like. These and other input devices connect to the processing unit 4604 through the system bus 4608 via interface port(s) 4630. Interface port(s) 4630 include, for example, a serial port, a parallel port, a game port, and a universal serial bus (USB). Output device(s) 4636 use some of the same type of ports as input device(s) 4628. Thus, for example, a USB port may be used to provide input to computer 4602, and to output information from computer 4602 to an output device 4636. Output adapter 4634 is provided to illustrate that there are some output devices 4636 like monitors, speakers, and printers, among other output devices 4636, which require special adapters. The output adapters 4634 include, by way of illustration and not limitation, video and sound cards that provide a means of connection between the output device 4636 and the system bus 4608. It should be noted that other devices and/or systems of devices provide both input and output capabilities such as remote computer(s) 4638.

Computer 4602 can operate in a networked environment using logical connections to one or more remote computers, such as remote computer(s) 4638. The remote computer(s) 4638 can be a personal computer, a server, a router, a network PC, a workstation, a microprocessor based appliance, a peer device, a smart phone, a tablet, or other network node, and typically includes many of the elements described relative to computer 4602. For purposes of brevity, only a memory storage device 4640 is illustrated with remote computer(s) 4638. Remote computer(s) 4638 is logically connected to computer 4602 through a network interface 4642 and then connected via communication connection(s) 4646. Network interface 4642 encompasses wire and/or wireless communication networks such as local-area networks (LAN) and wide-area networks (WAN) and cellular networks. LAN technologies include Fiber Distributed Data Interface (FDDI), Copper Distributed Data Interface (CDDI), Ethernet, Token Ring and the like. WAN technologies include, but are not limited to, point-to-point links, circuit switching networks like Integrated Services Digital Networks (ISDN) and variations thereon, packet switching networks, and Digital Subscriber Lines (DSL).

Communication connection(s) 4646 refers to the hardware/software employed to connect the network interface 4642 to the bus 4608. While communication connection 4646 is shown for illustrative clarity inside computer 4602, it can also be external to computer 4602. The hardware/software necessary for connection to the network interface 4642 includes, for exemplary purposes only, internal and external technologies such as, modems including regular telephone grade modems, cable modems and DSL modems, ISDN adapters, and wired and wireless Ethernet cards, hubs, and routers.

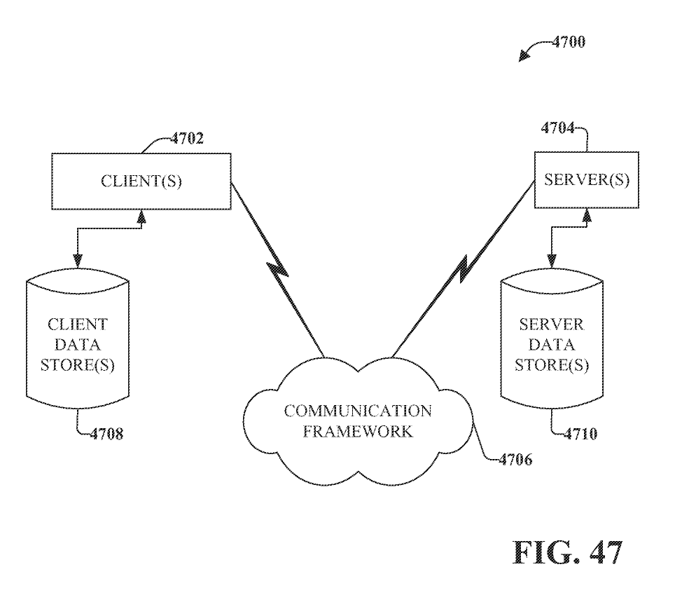

Referring now to FIG. 47, there is illustrated a schematic block diagram of a computing environment 4700 in accordance with this disclosure. The system 4700 includes one or more client(s) 4702 (e.g., laptops, smart phones, PDAs, media players, computers, portable electronic devices, tablets, and the like). The client(s) 4702 can be hardware and/or software (e.g., threads, processes, computing devices). The system 4700 also includes one or more server(s) 4704. The server(s) 4704 can also be hardware or hardware in combination with software (e.g., threads, processes, computing devices). The servers 4704 can house threads to perform transformations by employing aspects of this disclosure, for example. One possible communication between a client 4702 and a server 4704 can be in the form of a data packet transmitted between two or more computer processes wherein the data packet may include video data. The data packet can include a metadata, such as associated contextual information for example. The system 4700 includes a communication framework 4706 (e.g., a global communication network such as the Internet, or mobile network(s)) that can be employed to facilitate communications between the client(s) 4702 and the server(s) 4704.

Communications can be facilitated via a wired (including optical fiber) and/or wireless technology. The client(s) 4702 include or are operatively connected to one or more client data store(s) 4708 that can be employed to store information local to the client(s) 4702 (e.g., associated contextual information). Similarly, the server(s) 4704 are operatively include or are operatively connected to one or more server data store(s) 4710 that can be employed to store information local to the servers 4704.

In one embodiment, a client 4702 can transfer an encoded file, in accordance with the disclosed subject matter, to server 4704. Server 4704 can store the file, decode the file, or transmit the file to another client 4702. It is to be appreciated, that a client 4702 can also transfer uncompressed file to a server 4704 and server 4704 can compress the file in accordance with the disclosed subject matter. Likewise, server 4704 can encode video information and transmit the information via communication framework 4706 to one or more clients 4702.

The illustrated aspects of the disclosure may also be practiced in distributed computing environments where certain tasks are performed by remote processing devices that are linked through a communications network. In a distributed computing environment, program modules can be located in both local and remote memory storage devices.

Moreover, it is to be appreciated that various components described in this description can include electrical circuit(s) that can include components and circuitry elements of suitable value in order to implement the embodiments of the subject innovation(s). Furthermore, it can be appreciated that many of the various components can be implemented on one or more integrated circuit (IC) chips. For example, in one embodiment, a set of components can be implemented in a single IC chip. In other embodiments, one or more of respective components are fabricated or implemented on separate IC chips.

What has been described above includes examples of the embodiments of the present invention. It is, of course, not possible to describe every conceivable combination of components or methodologies for purposes of describing the claimed subject matter, but it is to be appreciated that many further combinations and permutations of the subject innovation are possible. Accordingly, the claimed subject matter is intended to embrace all such alterations, modifications, and variations that fall within the spirit and scope of the appended claims. Moreover, the above description of illustrated embodiments of the subject disclosure, including what is described in the Abstract, is not intended to be exhaustive or to limit the disclosed embodiments to the precise forms disclosed. While specific embodiments and examples are described in this disclosure for illustrative purposes, various modifications are possible that are considered within the scope of such embodiments and examples, as those skilled in the relevant art can recognize.

In particular and in regard to the various functions performed by the above described components, devices, circuits, systems and the like, the terms used to describe such components are intended to correspond, unless otherwise indicated, to any component which performs the specified function of the described component (e.g., a functional equivalent), even though not structurally equivalent to the disclosed structure, which performs the function in the disclosure illustrated exemplary aspects of the claimed subject matter. In this regard, it will also be recognized that the innovation includes a system as well as a computer-readable storage medium having computer-executable instructions for performing the acts and/or events of the various methods of the claimed subject matter.

The aforementioned systems/circuits/modules have been described with respect to interaction between several components/blocks. It can be appreciated that such systems/circuits and components/blocks can include those components or specified sub-components, some of the specified components or sub-components, and/or additional components, and according to various permutations and combinations of the foregoing. Sub-components can also be implemented as components communicatively coupled to other components rather than included within parent components (hierarchical). Additionally, it should be noted that one or more components may be combined into a single component providing aggregate functionality or divided into several separate sub-components, and any one or more middle layers, such as a management layer, may be provided to communicatively couple to such sub-components in order to provide integrated functionality. Any components described in this disclosure may also interact with one or more other components not specifically described in this disclosure but known by those of skill in the art.

In addition, while a particular feature of the subject innovation may have been disclosed with respect to only one of several implementations, such feature may be combined with one or more other features of the other implementations as may be desired and advantageous for any given or particular application. Furthermore, to the extent that the terms “includes,” “including,” “has,” “contains,” variants thereof, and other similar words are used in either the detailed description or the claims, these terms are intended to be inclusive in a manner similar to the term “comprising” as an open transition word without precluding any additional or other elements.

As used in this application, the terms “component,” “module,” “system,” or the like are generally intended to refer to a computer-related entity, either hardware (e.g., a circuit), a combination of hardware and software, software, or an entity related to an operational machine with one or more specific functionalities. For example, a component may be, but is not limited to being, a process running on a processor (e.g., digital signal processor), a processor, an object, an executable, a thread of execution, a program, and/or a computer. By way of illustration, both an application running on a controller and the controller can be a component. One or more components may reside within a process and/or thread of execution and a component may be localized on one computer and/or distributed between two or more computers. Further, a “device” can come in the form of specially designed hardware; generalized hardware made specialized by the execution of software thereon that enables the hardware to perform specific function; software stored on a computer readable storage medium; software transmitted on a computer readable transmission medium; or a combination thereof.

Moreover, the words “example” or “exemplary” are used in this disclosure to mean serving as an example, instance, or illustration. Any aspect or design described in this disclosure as “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

Computing devices typically include a variety of media, which can include computer-readable storage media and/or communications media, in which these two terms are used in this description differently from one another as follows. Computer-readable storage media can be any available storage media that can be accessed by the computer, is typically of a non-transitory nature, and can include both volatile and nonvolatile media, removable and non-removable media. By way of example, and not limitation, computer-readable storage media can be implemented in connection with any method or technology for storage of information such as computer-readable instructions, program modules, structured data, or unstructured data. Computer-readable storage media can include, but are not limited to, RAM, ROM, EEPROM, flash memory or other memory technology, CD-ROM, digital versatile disk (DVD) or other optical disk storage, magnetic cassettes, magnetic tape, magnetic disk storage or other magnetic storage devices, or other tangible and/or non-transitory media which can be used to store desired information. Computer-readable storage media can be accessed by one or more local or remote computing devices, e.g., via access requests, queries or other data retrieval protocols, for a variety of operations with respect to the information stored by the medium.

On the other hand, communications media typically embody computer-readable instructions, data structures, program modules or other structured or unstructured data in a data signal that can be transitory such as a modulated data signal, e.g., a carrier wave or other transport mechanism, and includes any information delivery or transport media. The term “modulated data signal” or signals refers to a signal that has one or more of its characteristics set or changed in such a manner as to encode information in one or more signals. By way of example, and not limitation, communication media include wired media, such as a wired network or direct-wired connection, and wireless media such as acoustic, RF, infrared and other wireless media.

In view of the exemplary systems described above, methodologies that may be implemented in accordance with the described subject matter will be better appreciated with reference to the flowcharts of the various figures. For simplicity of explanation, the methodologies are depicted and described as a series of acts. However, acts in accordance with this disclosure can occur in various orders and/or concurrently, and with other acts not presented and described in this disclosure. Furthermore, not all illustrated acts may be required to implement the methodologies in accordance with certain aspects of this disclosure. In addition, those skilled in the art will understand and appreciate that the methodologies could alternatively be represented as a series of interrelated states via a state diagram or events. Additionally, it should be appreciated that the methodologies disclosed in this disclosure are capable of being stored on an article of manufacture to facilitate transporting and transferring such methodologies to computing devices. The term article of manufacture, as used in this disclosure, is intended to encompass a computer program accessible from any computer-readable device or storage media.

What is claimed is:

1. A device for detecting biological targets comprising:
a memory having stored thereon computer executable components;
and a processor configured to execute the following computer executable components stored in the memory:
a cartridge component that stores nanomaterial labels paired to biological materials in N wells, whereby each well stores one or more nanomaterial labels paired to one or more biological materials; wherein N is an integer;
a dispenser component that dispenses a biological sample into each respective well;
an excitation component that emits energy from an excitation source to excite the nanomaterial label in the absence of the biological sample and excites the nanomaterial label in the presence of the biological sample;
a detection component that detects a relative change in energy emitted from the nanomaterial label paired to the biological material in the absence of the biological sample as compared to the energy emitted from the nanomaterial label paired to a biological material in the presence of a biological target present in the biological sample;
an analysis component that analyzes data related to the change in energy emitted from the nanomaterial label paired to the biological material as compared to a standard curve for the energy emitted from the nanomaterial label paired to a the biological material in the presence of various concentration levels of the biological target;
a generation component that generates a medical diagnostic report based in part on the analyzed data;
a display component that displays the medical diagnostic report to a device user.

2. The device of claim 1, wherein the analysis component employs software component to compute the concentration of one or more biological targets in a biological sample.

3. The device of claim 1, further comprising a synchronization component that synchronizes the device to a network system.

4. The device of claim 3, wherein the network system is an electronic medical record system.

5. The device of claim 1, wherein the biological material is any one or more of a: analyte, antibody, enzyme, nucleic acid, protein, polysaccharide, small molecule, avidin, streptavidin, biotin, antidigoxiginen, monoclonal antibody, polyclonal antibody, nucleic acid, monomeric nucleic acid, oligomeric nucleic acid, protein, sugar, peptide, drug, carbohydrate, DNA RNA, or glucose oxidase.

6. The device of claim 1, wherein the biological sample is any of cells, tissue, fluid, blood, plasma, serum, spinal fluid, cerebrospinal fluid, vaginal fluid, ascetic fluid, semen, lymph fluid, the external sections of the skin, respiratory sample, intestinal sample, genitourinary tract sample, tears, saliva, sputum, stool, throat swab, mucous, urine, blood, milk, blood cells, tumors, organs, in vitro cell cultures, or in vivo cell cultures.

7. The device of claim 1, wherein the nanomaterial label is a luminescent quantum dot.

8. The device of claim 7, wherein the luminescent quantum dot is at least one of: a spherical quantum dot, tetrapod quantum dot, quantum dot heterostructure, or luminescent multi-leg nanomaterial.

9. The device of claim 1, configured to operate on any of a mobile device, tablet computer, laptop computer, desktop computer, digital pen, medical testing tool or electronic device.

10. The device of claim 1, wherein the biological target is at least one of: glucose, sodium, calcium, chloride, potassium, albumin, alkaline phosphates (ALP), alanine aminotransferase (ALT), aspartate aminotransferase (AST), blood urea nitrogen (BUN), bicarbonate, magnesium, carbon dioxide (CO2), creatinine, bilirubin, protein, amylase, cholesterol, HDL cholesterol, LDL cholesterol, Cortisol, creatine kinase, creatanine kinase, estriol, ferritin, follicle-stimulating hormone, pH gas, PCo2 gas, Po2 gas, growth hormone-arginine stimulation, immunoglobulins, IgA, IgE, IgG, IgM, Iron, Lactate dehydrogenase, Luteinizing hormone, osmolality, parathyroid hormone, Prolactin (hPRL), globulin, thyroid-stimulating hormone, throidal iodine uptake, thyroxine, Triglycerides, triiodothyronine, triiodothyronine resin uptake, or ureic acid.

11. The device of claim 1, wherein the cartridge component is disposable.

12. A method, comprising:
using a processor to execute the following computer executable instructions stored in a memory to perform the following acts:
storing nanomaterial labels paired to biological materials in N wells, whereby each well stores one or more nanomaterial labels paired to one or more biological materials; wherein N is an integer;
dispensing a biological sample into each respective well;
emitting energy from an excitation source to excite the nanomaterial label in the absence of the biological sample and excites the nanomaterial label in the presence of the biological sample;
detecting a relative change in energy emitted from the nanomaterial label paired to the biological material in the absence of the biological sample as compared to the energy emitted from the nanomaterial label paired to a biological material in the presence of a biological target present in the biological sample;
analyzing data related to the change in energy emitted from the nanomaterial label paired to the biological material as compared to a standard curve for the energy emitted from the nanomaterial label paired to the biological material in the presence of various concentration levels of the biological target;
generating a medical diagnostic report based in part on the analyzed data;
displaying the medical diagnostic report to a device user.

13. The method of claim 12, comprising computing the concentration of one or more biological targets in a biological sample.

14. The method of claim 12, comprising synchronizing to a network system.

15. The method of claim 14, wherein the network system is an electronic medical record system.

16. The method of claim 12, wherein the biological material is any one or more of a: analyte, antibody, enzyme, nucleic acid, protein, polysaccharide, small molecule, avidin, streptavidin, biotin, antidgoxiginen, monoclonal antibody, polyclonal antibody, nucleic acid, monomeric nucleic acid, oligomeric nucleic acid, protein, sugar, peptide, drug, carbohydrate, DNA RNA, or glucose oxidase.

17. The method of claim 12, wherein the biological sample is any of cells, tissue, fluid, blood, plasma, serum, spinal fluid, cerebrospinal fluid, vaginal fluid, ascetic fluid, semen, lymph fluid, the external sections of the skin, respiratory sample, intestinal sample, genitourinary tract sample, tears, saliva, sputum, stool, throat swab, mucous, urine, blood, milk, blood cells, tumors, organs, in vitro cell cultures, or in vivo cell cultures.

18. The method of claim 12, wherein the nanomaterial label is a luminescent quantum dot.

19. The method of claim 12, wherein the luminescent quantum dot is at least one of: a spherical quantum dot, tetrapod quantum dot, quantum dot heterostructure, or luminescent multi-leg nanomaterial.

20. The method of claim 12, wherein the method is implemented on any of a mobile device, tablet computer, laptop computer, desktop computer, digital pen, medical testing tool or electronic device.
